(19) United States
(12) Patent Application Publication
(10) Pub. No.: US 2023/0063196 A1
(43) Pub. Date: Mar. 2, 2023

(54) INTRAVASCULAR BLOOD PUMP IN COMBINATION WITH CATHETER CONFIGURED TO CONTROL PUMP POSITION IN PATIENT'S HEART

(71) Applicant: ABIOMED, Inc., Danvers, MA (US)

(72) Inventors: Gerd Bruno Spanier, Aachen (DE); Joerg Schumacher, Aachen (DE); Christopher Zarins, Danvers, MA (US); Ralph Louis D'Ambrosio, Danvers, MA (US)

(73) Assignee: ABIOMED, Inc., Danvers, MA (US)

(21) Appl. No.: 17/899,022

(22) Filed: Aug. 30, 2022

Related U.S. Application Data
(60) Provisional application No. 63/238,999, filed on Aug. 31, 2021.
(60) Provisional application No. 63/245,308, filed on Sep. 17, 2021.

(51) Int. Cl. A61M 60/13; A61M 60/414; A61M 60/237; A61M 60/829; A61M 60/81

(52) U.S. Cl. CPC A61M 60/13; A61M 60/414; A61M 60/237; A61M 60/829; A61M 60/81

(57) ABSTRACT

Drive components and rotor housings for use in intravascular blood pumps, such as blood pumps configured to make the pump section more resistant to bending, kinking, and/or plastic deformation in combination with a catheter that controls a position of the intravascular blood pump to mitigate suction events that may occur due to the pump section's proximity to a patient's vasculature.

### CROSS-REFERENCE TO RELATED APPLICATIONS

This application claims priority to, and the benefit of, U.S. Provisional Application No. 63/238,999, filed Aug. 31, 2021, and U.S. Provisional Application No. 63/245,308, filed Sep. 17, 2021, the entire disclosures of which are hereby incorporated by reference herein.

### BACKGROUND

Intravascular blood pumps may be introduced into a patient either surgically or percutaneously and used to deliver blood from one location in the heart or circulatory system to another location in the heart or circulatory system. For example, when deployed in the left heart, an intravascular blood pump may pump blood from the left ventricle of the heart into the aorta. Likewise, when deployed in the right heart, an intravascular blood pump may pump blood from the inferior vena cava into the pulmonary artery. Intravascular blood pumps may be powered by a motor located outside of the patient's body via an elongated drive shaft or by an onboard motor located inside the patient's body. Some intravascular blood pump systems may operate in parallel with the native heart to supplement cardiac output and partially or fully unload components of the heart.

An intravascular blood pump for percutaneous insertion is typically delivered into the patient tethered to a catheter. The catheter may extend along a longitudinal axis from a distal end to a proximal end, with the pumping device being attached to the catheter at the end remote (distal) from an operator, such as a surgeon. The pumping device may be inserted through the femoral artery or the aorta into the left ventricle of a patient's heart by operation of the catheter. The blood pumps are often provided with an atraumatic tip at their far distal end (i.e., distal of the pumping device). The atraumatic tip mitigates any damage to the patient's soft tissue as the blood pump is positioned into the patient's heart.

Once the blood pump is inserted into the patient's heart, the pumping device of the blood pump generally positions itself close to the ventricular wall (i.e., septum) or close to the mitral valve of the heart. Positioning of the pumping device is itself atraumatic to the patient's vasculature and the heart itself, but when the blood pump operates in this position it may cause suctioning to the walls of the heart, heart valves (e.g., the mitral valve), or any other anatomical structure in the heart. In addition, the pumping device positioned near the septum may generate vibrations to the pump-system, cannula and catheter, and such vibrations may induce heart arrythmias. While positioning the pumping device in the apex of the ventricle (away from the septum and mitral valve) is thought to alleviate the aforementioned issues, the positioning of the pumping device precisely in the apex of the ventricle is difficult to achieve.

Accordingly, there exists a need for a blood pump having a catheter configured to permit control of the position of the pumping device of the blood pump when inserted into a patient's heart.

### SUMMARY

The present technology relates to improved drive components and rotor housings for use in intravascular blood pumps, such as blood pumps configured to make the pump section more resistant to bending, kinking, and/or plastic deformation in combination with a catheter that controls a position of the intravascular blood pump to mitigate suction events caused by the proximity of the pump section to a patient's vasculature. In some embodiments, the disclosed intravascular blood pumps may include a motor located outside of the patient's body and a rotor is driven by a flexible drive shaft. The intravascular blood pumps also may be those with motors located inside the patient's body, those without expandable and compressible rotor housings, those with rigid drive shafts, those with shorter flexible drive shafts, etc.

In addition, described herein is a sleeve configured to control a position of a blood pump with a catheter in a patient's heart. The sleeve may include a plurality of annular rings, at least two connectors disposed between each of the plurality of annular rings for connecting each of the plurality of annular rings and a plurality of openings formed between each annular ring and arranged in a repeating and optionally in an alternating repeating fashion. The sleeve may be adapted to be monolithically integrated with or placed over a predefined bend region of the catheter that is on a proximal end of a pumping device of the blood pump.

Also described herein is a blood pump with the sleeve described above. The blood pump may include a catheter having a predefined bend region, a pumping device connected to the catheter, and a sleeve configured to control a position of the blood pump with the catheter in a patient's heart. The sleeve may be adapted to be monolithically integrated with or placed over a predefined bend region of the catheter that is on a proximal end of a pumping device of the blood pump.

In one aspect, the disclosure describes an intravascular blood pump, comprising: a catheter; a housing in which a rotor is housed, the housing being attached to a distal end of the catheter; and a drive shaft extending through the catheter and connected to the rotor, at least a portion of the drive shaft being flexible, the drive shaft comprising an outer layer of wound or braided wires, an inner layer of wound or braided wires, and a reinforcement element arranged within at least the outer layer of wound or braided wires, wherein the drive shaft is rotatably supported in a proximal bearing located proximal of the rotor and a distal bearing located distal of the rotor, and wherein the reinforcement element extends from at least a point within the proximal bearing to a point within the distal bearing. In some aspects, the reinforcement element extends from a point proximal to the proximal bearing to a point within the distal bearing. In some aspects, the proximal bearing comprises a bearing sleeve attached to the drive shaft and an outer bearing ring attached to the housing, the bearing sleeve being configured to rotate within the outer bearing ring. In some aspects, the intravascular blood pump further comprises a restriction element attached to the housing and located proximal of the proximal bearing and configured to prevent the bearing sleeve from becoming dislodged from the outer bearing ring. In some aspects, the reinforcement element comprises a stepped proximal end with a portion of reduced diameter, and a portion of increased diameter. In some aspects, the portion of reduced diameter extends from a point at or substantially near where the catheter is attached to the housing to a point within the restriction element. In some aspects, the portion of reduced diameter extends from a point within the restriction element to a point within the proximal bearing. In some aspects, the portion of increased diameter extends from a point within the restriction element to a point within the distal bearing. In some aspects, the inner layer of wound or braided wires is omitted between a point within the restriction element and a point within the distal bearing. In some aspects, the portion of increased diameter extends from a point within the proximal bearing to a point within the distal bearing. In some aspects, the inner layer of wound or braided wires is omitted between a point within the proximal bearing and a point within the distal bearing. In some aspects, the reinforcement element comprises Nitinol or Ultra-Stiff Nitinol. In some aspects, the housing comprises a cage surrounding the rotor, the cage having a plurality of struts. In some aspects, at a first point proximal of the rotor, each strut of the plurality of struts has a circumferential width and a radial thickness, the circumferential width being between 1.2 and 1.8 times the radial thickness. In some aspects, at a first point proximal of the rotor, each strut of the plurality of struts has a circumferential width and a radial thickness, the circumferential width being between 1.2 and 1.3 times the radial thickness. In some aspects, at a first point proximal of the rotor, each strut of the plurality of struts has a circumferential width and a radial thickness, the circumferential width being about 1.26 times the radial thickness. In some aspects, at a second point distal of the rotor, each strut of the plurality of struts has a circumferential width and a radial thickness, the circumferential width being between 1.2 and 1.8 times the radial thickness. In some aspects, at a second point distal of the rotor, each strut of the plurality of struts has a circumferential width and a radial thickness, the circumferential width being between 1.2 and 1.3 times the radial thickness. In some aspects, at a second point distal of the rotor, each strut of the plurality of struts has a circumferential width and a radial thickness, the circumferential width being about 1.26 times the radial thickness. In some aspects, at a third point proximal of the rotor and distal of the first point, each strut of the plurality of struts has a circumferential width and a radial thickness, the circumferential width being between 1.0 and 1.6 times the radial thickness. In some aspects, at a third point proximal of the rotor and distal of the first point, each strut of the plurality of struts has a circumferential width and a radial thickness, the circumferential width being between 1.0 and 1.15 times the radial thickness. In some aspects, at a third point proximal of the rotor and distal of the first point, each strut of the plurality of struts has a circumferential width and a radial thickness, the circumferential width being about 1.26 times the radial thickness. In some aspects, at a third point proximal of the rotor and distal of the first point, each strut of the plurality of struts has a circumferential width and a radial thickness, the circumferential width being about 1.09 times the radial thickness. In some aspects, at a fourth point distal of the rotor and proximal of the second point, each strut of the plurality of struts has a circumferential width and a radial thickness, the circumferential width being between 1.0 and 1.6 times the radial thickness. In some aspects, at a fourth point distal of the rotor and proximal of the second point, each strut of the plurality of struts has a circumferential width and a radial thickness, the circumferential width being between 1.0 and 1.15 times the radial thickness. In some aspects, at a fourth point distal of the rotor and proximal of the second point, each strut of the plurality of struts has a circumferential width and a radial thickness, the circumferential width being about 1.26 times the radial thickness. In some aspects, at a fourth point distal of the rotor and proximal of the second point, each strut of the plurality of struts has a circumferential width and a radial thickness, the circumferential width being about 1.09 times the radial thickness. In some aspects, the housing comprises Nitinol or Ultra-Stiff Nitinol. In some aspects, the portion of increased diameter is configured to fit within the outer layer of the wound or braided wires in a portion of the drive shaft in which the inner layer of wound or braided wires has been omitted.

In another aspect, the disclosure describes a blood pump comprising: (1) a catheter having a distal end and a predefined bend region positioned proximal to the distal end; (2) a pumping device connected to the distal end of the catheter; and (3) a sleeve configured to control a position of the pumping device in a patient's heart, the sleeve comprising: a plurality of annular rings; at least two connectors, the at least two connectors disposed between each annular ring for connecting each of the plurality of annular rings, the at least two connectors being offset from adjacent connectors; and a plurality of openings formed between each ring, wherein the sleeve is configured to be monolithically integrated with or placed over the predefined bend region of the catheter and thereby provide a predefined resilient bend in the catheter at the predefined bend region. In some aspects, the blood pump further comprises an atraumatic tip at a distal end of the blood pump. In some aspects, the predefined bend region of the catheter is adapted to make contact with an endothelium of an aorta when the blood pump is inserted into a patient's heart, thereby supporting the pumping device and aligning the atraumatic tip with an aortic valve of the patient's heart and to thereby position the pumping device in a ventricle of the patient's heart. In some aspects, the atraumatic tip is between 110 to 140 degrees out of plane with respect to a plane in which the sleeve, when bent, lies flat, optionally 120 to 130 degrees, and optionally 130 degrees. In some aspects, the plurality of openings are formed in radially matched pairs which define a semicircle of 180 degrees about a circumference of the sleeve. In some aspects, each of the openings extends approximately a half way around the circumference of the sleeve and each opening having a connector at an opening terminus. In some aspects, the radially matched pairs of openings share a common axis and are laterally offset from one another in an alternating fashion. In some aspects, the plurality of annular rings are spaced apart by a uniform distance when the sleeve is in a straight configuration. In some aspects, a length of the sleeve corresponds to a length of the predefined bend region on the catheter.

In another aspect, the disclosure describes a catheter sleeve comprising: a plurality of annular rings; at least two connectors disposed between each of the plurality of annular rings for connecting each of the plurality of annular rings, the at least two connectors being offset from at least one adjacent connector; and a plurality of openings formed between each annular ring and arranged in an alternating repeating fashion, wherein the sleeve is configured to be monolithically integrated with or placed over a predefined bend region of a catheter and thereby provide a predefined resilient bend in the catheter.

### BRIEF DESCRIPTION OF DRAWINGS

FIG. 1 depicts an exemplary intravascular blood pump positioned within a left ventricle of a heart, in accordance with aspects of the disclosure.

FIG. 2 depicts an exemplary intravascular blood pump, in accordance with aspects of the disclosure.

FIG. 3 depicts a cross-sectional view of an exemplary configuration of the proximal end of the pump section of an intravascular blood pump, in accordance with aspects of the disclosure.

FIGS. 4A and 4B depict cross-sectional views of an exemplary configuration of the pump section of an intravascular blood pump, in accordance with aspects of the disclosure.

FIGS. 5A and 5B depict cross-sectional views of an exemplary configuration of the pump section of an intravascular blood pump, in accordance with aspects of the disclosure.

FIG. 6A depicts a side view of an exemplary pump housing, in accordance with aspects of the disclosure.

FIG. 6B depicts a cross sectional view of the pump housing of FIG. 6A taken along the line A-A.

FIG. 7A illustrates an intravascular blood pump with a catheter being placed in a patient's heart through an aorta.

FIG. 7B illustrates an intravascular blood pump with a catheter and a sleeve placed thereon.

FIG. 7C is a bottom view of the intravascular blood pump with the catheter of FIG. 7B.

FIG. 8 illustrates a portion of the catheter of FIG. 7A with a sleeve placed thereon.

FIG. 9 is a perspective view of a first embodiment of the sleeve, which is configured to be used with the catheter of the intravascular blood pump of FIG. 7A.

FIG. 10 is another perspective view of the sleeve of FIG. 9.

FIG. 11 is a top view of the sleeve of FIG. 9.

FIG. 12 is a perspective view of a second embodiment of the sleeve, which is configured to be used with the catheter of the intravascular blood pump of FIG. 7A.

FIG. 13 is another perspective view of the sleeve of FIG. 12.

FIG. 14 is a top view of the sleeve of FIG. 12.

FIG. 15 is a perspective view of a third embodiment of the sleeve, which is configured to be used with the catheter of the intravascular blood pump of FIG. 7A.

FIG. 16 is another perspective view of the sleeve of FIG. 15.

FIG. 17 is a perspective view of a fourth embodiment of the sleeve, which is configured to be used with the catheter of the intravascular blood pump of FIG. 7A.

FIG. 18 is a perspective view of a fifth embodiment of the sleeve, which is configured to be used with the catheter of the intravascular blood pump of FIG. 7A.

FIG. 19 is a perspective view of a sixth embodiment of the sleeve, which is configured to be used with the catheter of the intravascular blood pump of FIG. 7A.

FIG. 20 is a side view of the sleeve of FIG. 19.

FIG. 21 is a perspective view of a seventh embodiment of the sleeve, which is configured to be used with the catheter of the intravascular blood pump of FIG. 7A.

FIG. 22 is a side view of the sleeve of FIG. 21.

FIG. 23 is a perspective view of an eight embodiment of the sleeve, which is configured to be used with the catheter of the intravascular blood pump of FIG. 7A.

FIG. 24 is a side view of the sleeve of FIG. 23.

FIG. 25 is a perspective view of a portion of a sleeve having a strain relief section according to some embodiments.

FIG. 26 is side view of the sleeve of strain relief section of the sleeve of FIG. 25.

FIG. 27 is a perspective view of a sleeve having a strain relief section according to another embodiments.

FIG. 28 is an enlarged side view of the strain relief section of FIG. 27.

FIG. 29 illustrates an intravascular blood pump with a catheter and a sleeve portion.

FIG. 30 illustrates another embodiment of an intravascular blood pump with a catheter and a sleeve portion.

FIG. 31 illustrates an intravascular blood pump with a catheter being placed in a patient's heart through the aorta.

FIG. 32 is another view of the blood pump of FIG. 27 placed in the patient's heart.

### DETAILED DESCRIPTION

The present technology will now be described with respect to certain exemplary systems, methods, and devices. In that regard, it is to be understood that the exemplary systems, methods, and devices disclosed herein are merely meant to illustrate examples of the present technology, which may be implemented in various forms. As such, well known functions or constructions are not described in detail to avoid obscuring the present disclosure in unnecessary detail. Likewise, specific structural and functional details disclosed herein are not to be interpreted as limiting, but merely as a basis for the claims and as a representative basis for teaching one skilled in the art to employ the present disclosure in other suitable structures. In that regard, although various examples may describe specific medical procedures and/or uses of intravascular blood pumps, it will be understood that the present technology may be employed in any suitable context.

As used herein, the terms “proximal” and “distal” refer to positions relative to a physician or operator of the intravascular blood pump. Thus, “proximal” indicates a position that is closer to the physician or operator or a direction that points towards the physician or operator, and “distal” indicates a position that is farther from the physician or operator or a direction that points away from the physician or operator. In addition, as used herein, the terms “bearing sleeve”, “outer sleeve”, and “sleeve” are three distinct terms. Specifically, the “bearing sleeve” and “outer sleeve” are structures disposed within the intravascular blood pump, whereas the “sleeve” is a structure positioned outside of the intravascular blood pump. In the present disclosure, reference numerals shared between figures are meant to identify similar or identical elements.

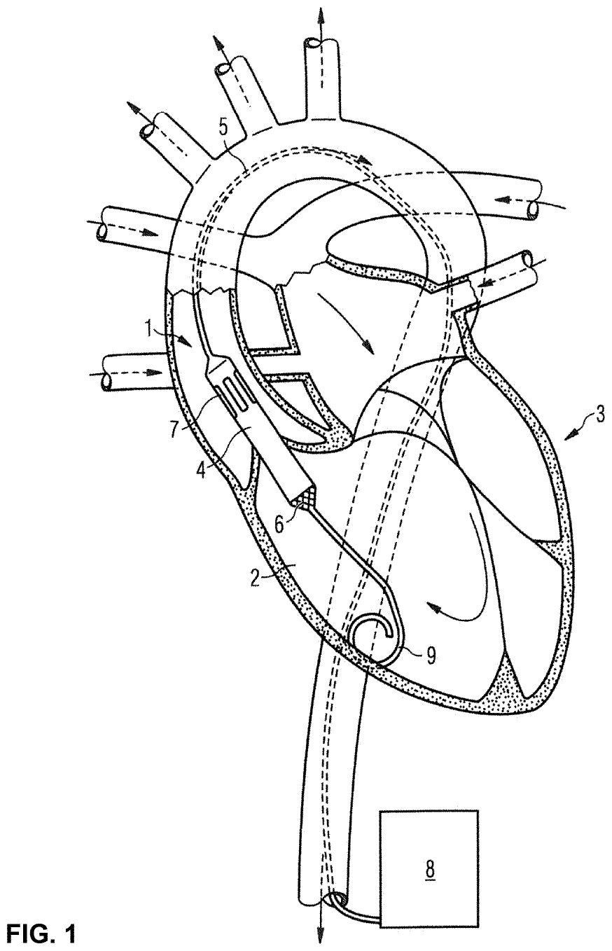

FIG. 1 shows an exemplary use of an intravascular blood pump 1 for supporting a left ventricle 2 of a human heart 3. The intravascular blood pump 1 may include a catheter 5 and a pump section 4 mounted at a distal end region of the catheter 5. The intravascular blood pump 1 may be placed inside the human heart 3 using a percutaneous, transluminal technique. For example, the intravascular blood pump 1 may be introduced through a femoral artery. Likewise, the intravascular blood pump 1 may be introduced through other vessels, such as through the subclavian artery. As shown in FIG. 1, the catheter 5 may be pushed into the aorta such that the pump section 4 reaches through the aortic valve into the heart.

The pump section 4 may further comprise a rotor (not visible in FIG. 1) to cause blood to flow from a blood flow inlet 6 at a distal end of the pump section 4 to a blood flow outlet 7 located proximally of the blood flow inlet 6. By placing the blood flow inlet 6 inside the left ventricle 2 and the blood flow outlet 7 inside the aorta, the intravascular blood pump 1 may support the patient's systemic blood circulation. If the intravascular blood pump 1 is configured and placed differently, it may be used, e.g., to support the patient's pulmonary blood circulation instead.

The catheter 5 may further house a drive shaft (not visible in FIG. 1) configured to be driven by an electric motor 8, which may be positioned outside the patient's body. The drive shaft may be configured to drive a rotor (not visible in FIG. 1) contained inside the pump section 4.

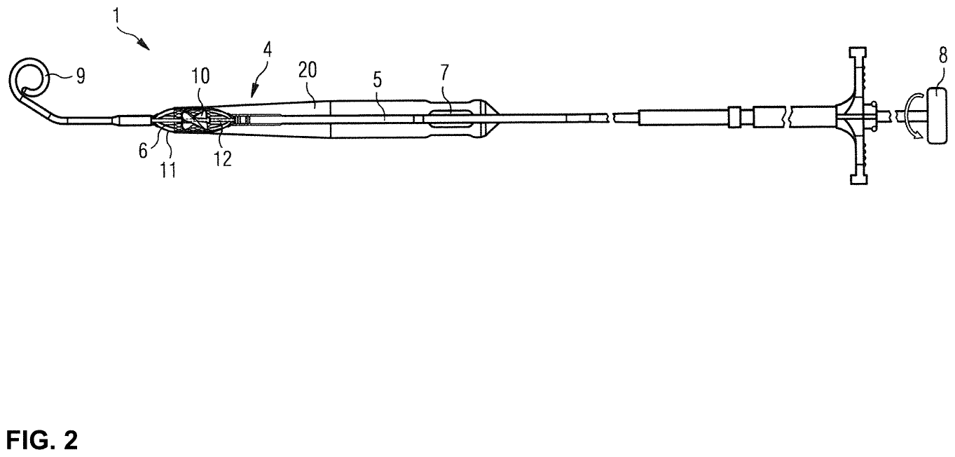

As shown in FIGS. 1 and 2, the pump section 4 may also have a flexible atraumatic tip 9 at its distal end. The flexible atraumatic tip 9 may have any suitable shape, such as a pigtail or a J-form, and may be configured to facilitate placement of the intravascular blood pump 1 by aiding navigation inside the patient's vascular system. Furthermore, the softness of the flexible atraumatic tip 9 may be configured to allow the pump section 4 to support itself atraumatically against a wall of the left ventricle 2.

FIG. 2 shows an exemplary intravascular blood pump 1 according to aspect of the disclosure. As shown in FIG. 2, a rotor 10 may be located inside a housing 11, and the housing 11 may form a cage around the rotor 10. Both the rotor 10 and the housing 11 may be made compressible, such that the intravascular blood pump 1 may be inserted into and/or through the patient's vascular system while both the rotor 10 and the housing 11 are in their compressed state, and such that the rotor 10 and housing 11 may be expanded once the pump section 4 is positioned at or near its target location in the patient's heart. For example, in some embodiments, expansion may occur when the housing 11 is in the ventricle, the ascending aorta, or the descending aorta. Likewise, in some embodiments, expansion may occur directly after the housing 11 is introduced into the patient's vasculature, with the housing 11 then being moved to its target location in the patient's heart in its expanded state. As will be appreciated, expansion may occur in any suitable location within the patient's vasculature, such as a portion of the patient's vasculature having a diameter that exceeds the diameter of the expanded housing 11. In some embodiments, the rotor 10 and housing 11 may be formed from any suitable material or materials. For example, in some aspects of the technology, the rotor 10 and/or housing 11 may be produced at least in part from polyurethane, silicone rubber, a shape-memory material such as Nitinol or Ultra-Stiff Nitinol (“USN”), etc.

The drive shaft 12 may extend through the entire catheter or only parts thereof. In some aspects, the drive shaft 12 may be hollow along all or a portion of its length. The drive shaft 12 or portions thereof may be formed from a cable, solid shaft, hollow shaft, or combinations thereof. In that regard, the drive shaft 12 may be a flexible cable formed of any suitable number of differently oriented fiber layers (e.g., 2 layers, 3 layers, 4 layers, etc.). For example, the drive shaft 12 may be formed from a plurality of coaxial windings, each with different or alternating winding directions. In such an example, the different or alternating winding directions may be running helically around a lumen extending axially along the drive shaft. In some aspects of the technology, the drive shaft 12 may include two coaxial windings, each with opposite winding directions, and an outer diameter of the drive shaft may be between 0.4 mm and 2 mm, preferably between 0.6 mm and 1.2 mm, particularly preferably between 0.8 mm and 1.0 mm. In cases where the drive shaft 12 has at least one outer layer and/or inner layer which includes a winding or windings, each wire of the winding may comprise one strand or several strands, e.g. that may be twisted. In some cases, the windings of a given layer may form a single helix. Likewise, in some cases, the windings of a given layer may include two or more helices which are preferably shifted axially, similar to a multistart thread. In some cases, the drive shaft 12 may include one or more layers of braided wire, similar to the outer sheath of a kernmantle rope. In all cases, the wire(s) of a given layer may be formed from any suitable metal or other material, and may further include one or more surface coatings.

In some aspects of the technology, a drive shaft 12 having one or more layers (e.g., as described herein) may be at least partly filled or coated with a sealant which penetrates into at least one layer. In some embodiments, such a sealant may be arranged to minimize and/or prevent penetration of fluids (e.g., purge fluid, bodily fluids) through the respective layers of the drive shaft. In some aspects, the sealant may penetrate into all layers. Any suitable sealant may be used in this regard. For example, in some aspects of the technology, the sealant may be selected based on its ability to penetrate into, between, and across the layers as a fluid and then harden. Any suitable material may be used as a sealant, such as adhesives, polymers, and/or thermoplastics.

In addition, in some aspects of the technology, a drive shaft 12 having one or more layers (e.g., as described herein) may be at least partly filled or coated with two or more different adhesives. Thus, in some aspects, a first adhesive or sealant may be used to penetrate one or more of the layers. For example, this first adhesive may be a sealant (as described herein), and may be selected to have a particularly low viscosity to enable it to penetrate the outer and/or the inner windings completely. In that regard, the first adhesive may have a viscosity in the range from 80 cPs to 200 cPs before hardening. A second adhesive may then be used to connect other members (e.g., the rotor 10, bearing sleeve 30 (see below), restriction member 33 (see below)) to the drive shaft 12. In some aspects of the technology, the second adhesive may have a higher viscosity than the first adhesive, and may thus have a paste-like consistency. In some cases, the first adhesive and second adhesive may both be two-part epoxy resins (of the same or different types).

As shown in the example of FIG. 2, the proximal end of the drive shaft 12 may be attached to an extracorporeal electric motor 8. In such a configuration, the drive shaft 12 may run through catheter 5, protrude from a distal end of the catheter 5, and serve to transfer torque from the electric motor 8 to the rotor 10 at the distal end of the drive shaft 12. In some aspects of the technology, the drive shaft 12 may include a stiff, rigid, and/or reinforced section at its distal end, onto which the rotor 10 is attached inside the housing 11, in order to provide stability to the rotor. Rotor 10 may be configured such that, when it is rotated by the drive shaft 12, blood is drawn into the blood flow inlet 6 at the distal end of the housing 11, and pumped through the housing 11 into a downstream tubing 20, which is attached to the housing 11 and extends proximally. The blood may then be ejected from the downstream tubing 20 through a blood flow outlet 7 provided in the downstream tubing 20. The blood flow outlet 7 may have a single opening, or any suitable number of openings.

In some aspects of the technology, the downstream tubing 20 may be made of a flexible material or materials such that it may be compressed by the aortic valve as the patient's heart is pumping. Likewise, in some aspects of the technology, the downstream tubing 20 may be configured to expand as a result of a blood flow generated by the rotor 10 during rotation.

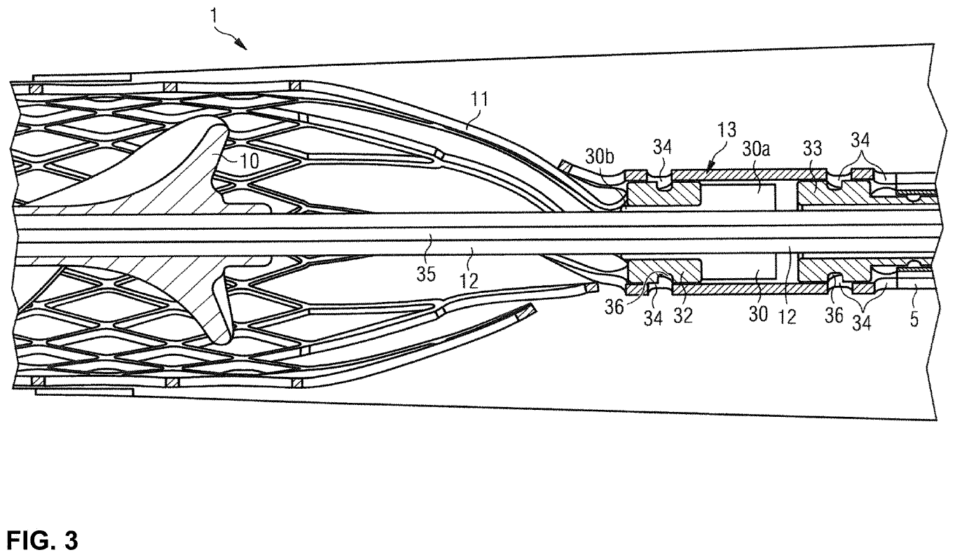

FIG. 3 depicts a cross-sectional view of an exemplary intravascular blood pump 1 with a housing 11, and a rotor 10 mounted on a drive shaft 12. The example of FIG. 3 employs a proximal bearing 13 arranged within the proximal end of housing 11. As shown in FIG. 3, the proximal bearing 13 may include a bearing sleeve 30 that is rotatably supported in an outer bearing ring 32. The bearing sleeve 30 may be fixed to the drive shaft 12 in any suitable way. For example, in some aspects of the technology, the drive shaft 12 may be bonded with bearing sleeve 30 using a suitable glue, weld, solder, or bonding material. Likewise, in some aspects, the bearing sleeve 30 may be crimped to or shrunk onto the drive shaft 12.

The bearing sleeve 30 and the outer bearing ring 32 may be formed from any suitable material or materials. For example, in some aspects of the technology, the bearing sleeve 30 and/or the outer bearing ring 32 may be formed from one or more ceramics. Likewise, in some aspects of the technology, the bearing sleeve 30 and/or the outer bearing ring 32 may be formed from one or more metals, such as MP35, 35NLT, Nitinol, or stainless steel. Further, where the bearing sleeve 30 and/or the outer bearing ring 32 are made from one or more metals, they may further include a hard coating, such as for example a coating made from diamond-like carbon (“DLC”).

Drive shaft 12 may take any of the forms described above with respect to FIG. 2 (e.g., flexible cable formed of any suitable number of differently oriented fiber layers). In the example of FIG. 3, the drive shaft 12 further includes a lumen in which a reinforcement element 35 is inserted. Reinforcement element 35 may be formed from any suitable material or materials, and may be configured in any suitable way. For example, in some aspects of the technology, reinforcement element 35 may be a solid rod or wire arranged coaxially within the drive shaft 12, e.g., made from spring steel, 1.4310 stainless steel, carbon wire, super-elastic or hyper-elastic materials like Nitinol, Ultra-Stiff Nitinol, etc. Likewise, in some aspects of the technology, the drive shaft 12 and/or reinforcement element 35 may be hollow along some or all of its length, such that it may also function as a conduit for purge fluid. For example, in some instances, the reinforcement element may include a hollow tube.

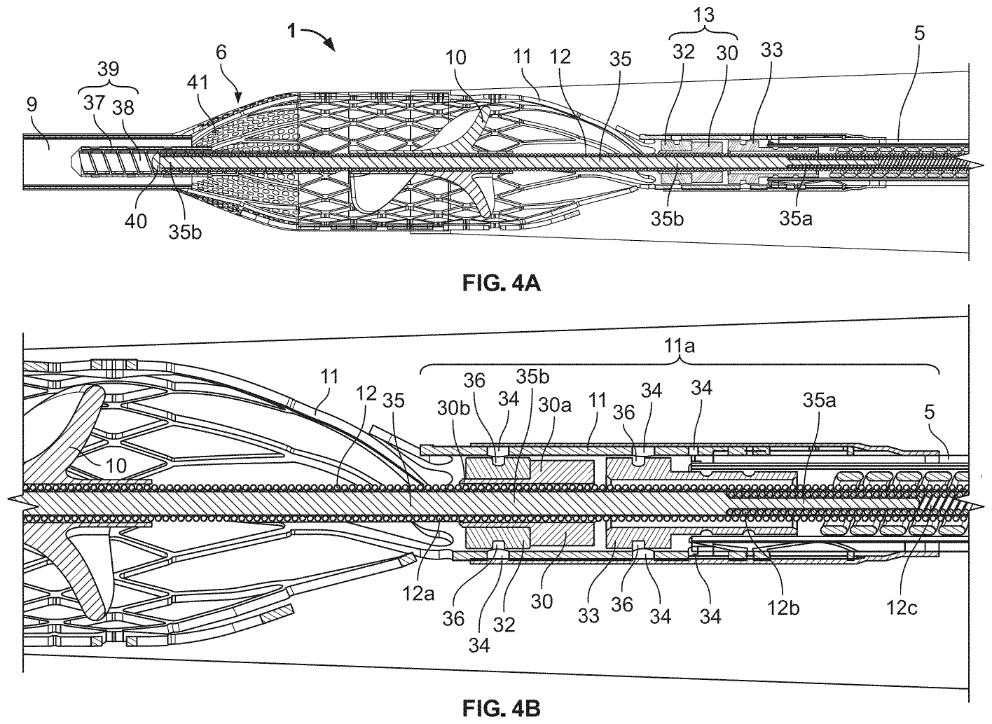

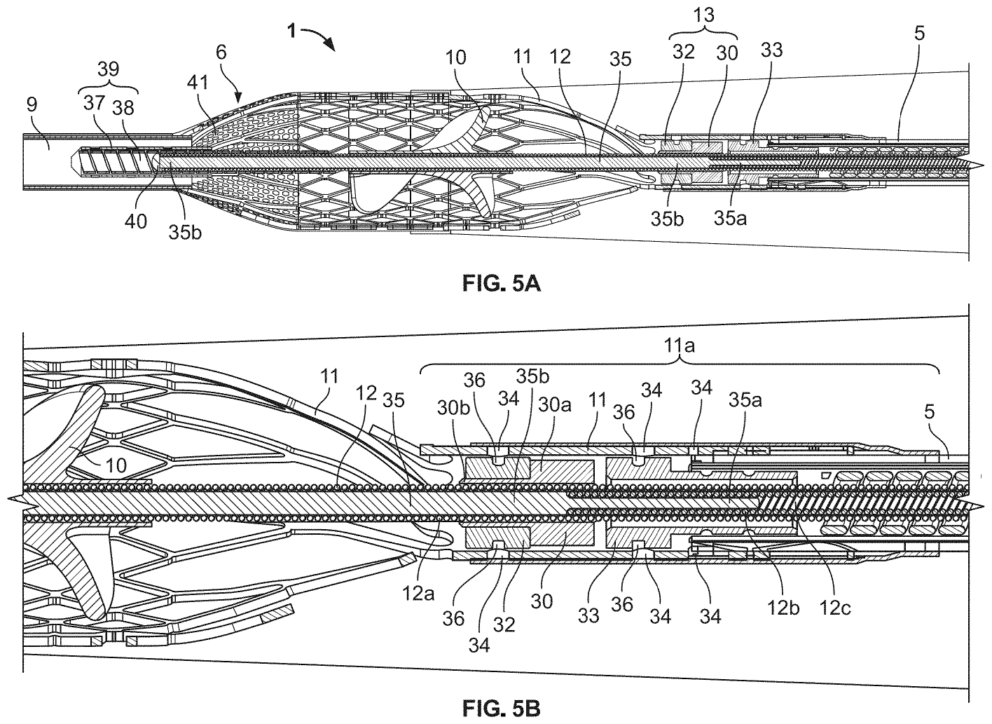

In addition, reinforcement element 35 may be any suitable length, and may be based on criteria including, but not necessarily limited to, optimizing stiffness of the pump section, preventing of plastic deformation during insertion, and/or reducing vibration during operation. For example, in some aspects of the technology, reinforcement element 35 may be configured to extend from a point proximal of the proximal bearing 13 to the distal end of the rotor 10 (not visible in FIG. 3). Likewise, in some aspects, reinforcement element 35 may be configured to extend from a point proximal of the proximal bearing 13 to a point within the distal bearing (not visible in FIG. 3), e.g., as shown and described below with respect to FIGS. 4A, 4B, 5A, and 5B. Further, the reinforcement element 35 may be configured to extend from a point at the proximal end of proximal bearing 13, or within the proximal bearing 13, to a point within the distal bearing.

As shown in FIG. 3, a restriction member 33 may be located proximal of the proximal end of the bearing sleeve 30 to strengthen the assembly and prevent the bearing sleeve 30 from backing away from and/or dislodging from the outer bearing ring 32. The restriction member 33 and the outer bearing ring 32 may be fixed to the bearing sleeve 30 in any suitable way. For example, in some aspects of the technology, the restriction member 33 and the outer bearing ring 32 may be press-fit into the proximal end of housing 11. Likewise, in some aspects, the restriction member 33 and the outer bearing ring 32 may be bonded with the proximal end of housing 11 using a suitable glue, weld, solder, or bonding material. Further, the restriction member 33 may also be fixed to the catheter 5 in any suitable way. Thus, in some aspects of the technology, the restriction member 33 may be press-fit into the catheter 5, or bonded with catheter 5 using a suitable glue, weld, solder, or bonding material. In this way, the restriction member 33 may also function to connect the housing 11 and the catheter 5.

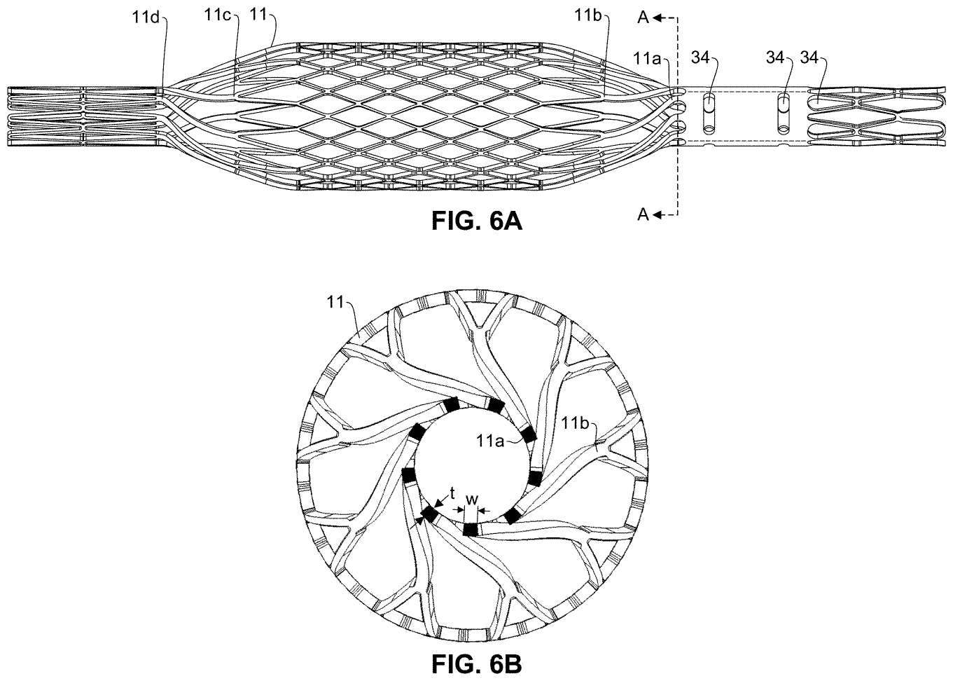

As shown in FIG. 3, the proximal end of housing 11 may include one or more through-holes 34. In some embodiments, the through-holes 34 may have any suitable shape and/or dimension. For example, in some aspects of the technology, through-holes 34 may be round holes with a suitable diameter (e.g., between 0.5 mm and 1 mm). Further, in some aspects of the technology, through-holes 34 may have a grooved shape that extends in a circumferential direction, e.g., as shown in the left-most and middle through-holes 34 of FIG. 6A. Likewise, in some aspects of the technology, through-holes 34 may be the holes of a diamond pattern, e.g., as shown in the right-most through-hole 34 of FIG. 6A. In addition, the outer bearing ring 32 and/or restriction member 33 may also each include one or more depressions or grooves 36 corresponding to one of the through-holes 34.

Through-holes 34 may increase elasticity of the proximal end of housing 11 to enable press-fitting of the outer bearing ring 32 and/or restriction member 33 within housing 11. In addition, through-holes 34 and corresponding depressions/grooves 36 may be used during manufacturing to confirm that the outer bearing ring 32 and/or the restriction member 33 have been positioned appropriately (e.g., such that a gap remains between the proximal end of outer bearing ring 32 and the distal end of restriction member 33).

Further, through-holes 34 may be used to allow a glue, weld, solder, or bonding material to be applied to fixedly connect the outer bearing ring 32 and/or the restriction member 33 to the housing 11. In such cases, the depressions/grooves 36 in the outer bearing ring 32 and/or restriction member 33 may also be configured to accept any glue, weld, solder, or bonding material applied through through-holes 34, and/or to aid in allowing it to flow within the proximal end of housing 11 to increase the surface area of the resulting bond. In some aspects of the technology, it may be advantageous to ensure that a glue, weld, solder, bonding material, or a further sealant fills the entirety of any through-holes 34 and/or depressions/grooves 36 to ensure that fluid may not enter or exit through them. For example, in cases where a purge fluid is to be applied to the proximal bearing 13, filling and/or sealing of through-holes 34 and grooves 36 may serve to prevent leakage of purge fluid intended to flow between the bearing sleeve 30 and the outer bearing ring 32.

As may be seen from FIG. 3, the bearing sleeve 30 comprises a proximal portion 30a located proximally of the outer bearing ring 32 and a distal portion 30b extending from the proximal portion 30a distally into the outer bearing ring 32. The proximal portion 30a forms an axial bearing with a proximal surface of the outer bearing 32, whereas the distal portion 30b forms a radial bearing with a radial inner surface of the outer bearing ring 32. In this way, in the example of FIG. 3, the proximal bearing 13 includes both an axial bearing and a radial bearing. However, as will be understood, in some aspects of the technology, the bearing sleeve 30 may be configured such that it will not contact any proximal surface of the outer bearing ring 32, in which case proximal bearing 13 may include only a radial bearing between the distal portion 30b of the bearing sleeve 30 and a radial inner surface of the outer bearing ring 32.

In some aspects of the technology, the intravascular blood pump 1 may be configured to supply a purge fluid to the proximal bearing 13, e.g., for purposes of lubrication and/or cooling. In such cases, purge fluid may be pumped through the proximal bearing 13 in a distal direction such that it first passes over the proximal portion 30a of the bearing sleeve 30 along a radial outer surface thereof, then flows radially inwards between the distal surface of the proximal portion 30a and the proximal surface of the outer bearing ring 32, and then flows in a distal direction between the distal portion 30b of the bearing sleeve 30 and the radial inner surface of the outer bearing ring 32. The bearing gaps between the distal surface of the proximal portion 30a and the proximal surface of the outer bearing ring 32, and between the distal portion 30b of the bearing sleeve 30 and the radial inner surface of the outer bearing ring 32, may be configured so that the purge fluid will flow through the bearing gaps in a closely controllable manner when suitable pressure is applied. For example, in some aspects of the technology, the bearing gap between the distal portion 30b of the bearing sleeve 30 and the radial inner surface of outer bearing ring 32 may be between 1 μm and 10 μm wide, for example between 2 μm and 8 μm wide, such as 3.5 μm wide.

Further, in some aspects of the technology, a radial notch or radial notches (not shown) may be provided in the proximal surface of the static outer bearing ring 32 to provide further space for purge fluid to flow in cases where the bearing sleeve 30 is pulled in a distal direction. For example, in some aspects of the technology, the rotor 10 and/or drive shaft 12 may be configured such that, during operation, the rotor 10 will have a tendency to pull and/or wind the drive shaft 12 such that the bearing sleeve 30 will move in a distal direction and thus press against the proximal surface of the outer bearing ring 32.

FIGS. 4A and 4B depict cross-sectional views of an exemplary configuration of the pump section of an intravascular blood pump, in accordance with aspects of the present disclosure. For example, FIG. 4A depicts a portion of the distal end of intravascular blood pump 1, and FIG. 4B shows an enlarged view of the proximal end of housing 11. Except as described in detail below, elements in FIGS. 4A and 4B that share the same reference numerals as those of FIGS. 1-3 are meant to identify the same structures described above. As such, any of the features and options discussed above with respect to such elements may likewise apply to the exemplary configuration of FIGS. 4A and 4B.

In the example of FIGS. 4A and 4B, reinforcement element 35 has a stepped proximal end with a portion of reduced diameter 35a, and a portion of increased diameter 35b extending from a point within restriction member 33 to the distal end of drive shaft 12. The drive shaft 12 may include an outer layer 12a of wound or braided wires, an inner layer 12b of wound or braided wires, and a lumen 12c. In FIG. 4A, both the proximal end of flexible atraumatic tip 9 and the distal bearing 39 are visible. In this example, distal bearing 39 may include an outer sleeve 37 which houses a spiral bearing 38, with spiral bearing 38 being configured to surround the drive shaft 12. FIG. 4A also shows an optional mesh 41 situated over the blood flow inlet 6. In addition, in some embodiments, another spiral bearing may also surround a portion of the drive shaft 12 proximal of the restriction member 33. For example, a spiral bearing may surround the drive shaft 12 from a point at or near the proximal end of the housing 11 to a point at or near the proximal end of the catheter 5, and may be configured to prevent the drive shaft 12 from rubbing against an inner surface of catheter 5 as it rotates.

In some embodiments, the portion of reduced diameter 35a may begin and end anywhere within the proximal section 11a of the housing 11. For example, as shown in FIGS. 4A and 4B, the portion of reduced diameter 35a at the proximal end of reinforcement element 35 may extend from a point at or near (e.g., substantially near) where the catheter 5 is coupled to the proximal end of housing 11 to a point within restriction member 33. However, as will be understood, in some aspects of the technology, the portion of reduced diameter 35a may begin at a point distal of where the catheter 5 is coupled to the proximal end of housing 11, and may extend to a point proximal or distal of the restriction member 33. Further, as shown in FIGS. 4A and 4B, this portion of reduced diameter 35a may be configured to be inserted within lumen 12c, while the portion of increased diameter 35b may be configured to fit within outer layer 12a in a portion of drive shaft 12 in which inner layer 12b has been omitted.

As will be appreciated, where drive shaft 12 includes more than two layers of windings, a one-step reinforcement element like that shown in FIGS. 4A and 4B may be arranged such that its portion of reduced diameter 35a and portion of increased diameter 35b are surrounded by any suitable combination of winding layers. For example, in some aspects, for a drive shaft having n layers, the portion of reduced diameter 35a be surrounded by innermost layer 1 and the portion of increased diameter 35b may be surrounded by layers 2 through n. Likewise, in some aspects, for a drive shaft having three layers, the portion of reduced diameter 35a may be surrounded by layer 2 and the portion of increased diameter 35b may be surrounded by outermost layer 3. Further, in some aspects, for a drive shaft having three layers, the portion of reduced diameter 35a may be surrounded by innermost layer 1 and the portion of increased diameter 35b may be surrounded by outermost layer 3, such that there is a larger step between the portion of reduced diameter 35a and the portion of increased diameter 35b. As will also be appreciated, where drive shaft 12 includes more than two layers of windings, a reinforcement element may also be configured with more than one step. Thus, for example, for a drive shaft having three layers, a two-step reinforcement element may be used, with its the narrowest portion being surrounded by layer 1, its next widest portion being surrounded by layer 2, and its widest portion being surrounded by layer 3.

Further, in some aspects of the technology, the proximal end of the portion of reduced diameter 35a also may begin at a point that is proximal to the proximal end of housing 11 or that is proximal of where the catheter 5 is coupled to the proximal end of housing 11 (e.g., proximal to an area of polymer reinforcement (not shown) on the outer circumference of the catheter 5, in which the assembly may be stiffer), and may extend to a point distal of the area of where the catheter 5 is coupled to the proximal end of housing 11 (e.g., distal to such an area of polymer reinforcement on the outer circumference of the catheter 5).

In some applications, the reinforcing arrangement shown in FIGS. 4A and 4B may allow the portion of increased diameter 35b to be thicker than lumen 12c, thus increasing stiffness in that portion of drive shaft 12 relative to what could be achieved with a reinforcement element of smaller outer diameter (e.g., as shown in the example of FIG. 3). In some embodiments, this may allow reinforcement element 35 to be manufactured from materials that may otherwise be too flexible and/or soft if the entirety of reinforcement element 35 had to fit within lumen 12c. The present technology may thus open up the option of reinforcing the drive shaft 12 with materials such as Nitinol and Ultra-Stiff Nitinol, which are particularly resistant to plastic deformation due to their hyper-elasticity, and yet may remain stiff enough (when reinforcement element 35 is configured as shown in FIGS. 4A and 4B) to control vibration and prevent rotor 10 from contacting housing 11.

In addition to the above, the stepped proximal end of reinforcement element 35 may provide for a more gradual transition in stiffness between the unreinforced and fully reinforced portions of drive shaft 12, which may make the drive shaft 12 more resistant to kinking at or near the proximal end of the reinforcement element 35. Further, the portion of reduced diameter 35a may provide an interface between reinforcement element 35 and inner layer 12b which may facilitate bonding. In that regard, in some aspects of the technology, reinforcement element 35 may be fixed within drive shaft 12 using a suitable glue, weld, solder, or other suitable bonding material (not shown). Likewise, as shown in FIGS. 4A and 4B, the distal end of reinforcement element 35 may be fixed to the distal end of drive shaft 12 using a suitable glue, weld, solder, or other suitable bonding material 40.

FIGS. 5A and 5B likewise depict cross-sectional views of an exemplary configuration of the pump section of an intravascular blood pump, in accordance with aspects of the disclosure. In particular, FIG. 5A depicts a portion of the distal end of intravascular blood pump 1, and FIG. 5B shows an enlarged view of the proximal end of housing 11. Except as described in detail below, elements in FIGS. 5A and 5B that share the same reference numerals as those of FIGS. 1-4B are meant to identify the same structures described above. As such, any of the features and options discussed above with respect to such elements may likewise apply to the exemplary configuration of FIGS. 5A and 5B.

As in FIGS. 4A and 4B, the example of FIGS. 5A and 5B also includes a reinforcement element 35 with a stepped proximal end. Here as well, the portion of reduced diameter 35a may begin and end anywhere within the proximal section 11a of the housing 11. Thus, as shown in the example of FIGS. 5A and 5B, the portion of reduced diameter 35a may extend from a point within restriction member 33 to a point within proximal bearing 13, and the portion of increased diameter 35b extends from a point within proximal bearing 13 to the distal end of drive shaft 12. However, as will be understood, in some aspects of the technology, the portion of reduced diameter 35a may begin proximal or distal of the restriction member 33, and may extend to a point proximal or distal of the proximal bearing 13. Here as well, the portion of reduced diameter 35a may be configured to be inserted within lumen 12c, while the portion of increased diameter 35b may be configured to fit within outer layer 12a in a portion of drive shaft 12 in which inner layer 12b has been omitted. The arrangement of FIGS. 5A and 5B thus may provide the same advantages discussed above with respect to FIGS. 4A and 4B. However, by locating the transition between the portion of reduced diameter 35a and the portion of increased diameter 35b within proximal bearing 13, and by locating the proximal end of the reinforcement member 35 within restriction member 33, the example shown in FIGS. 5A and 5B may also reduce bending of these portions of the drive shaft 12, and thus further resist kinking.

FIG. 6A depicts a side view of an exemplary pump housing, in accordance with aspects of the disclosure. FIG. 6B depicts a cross sectional view of the pump housing of FIG. 6A taken along the line A-A.

The exemplary pump housing 11 of FIGS. 6A and 6B may be used with any of the examples depicted and/or described herein. In this example, the housing 11 may include struts with circumferential widths that are larger than their radial thicknesses. For example, in some aspects of the technology, at point 11a, the strut may have a circumferential width w that is between about 1.2 and 1.8 times the radial thickness t. For example, in some embodiments, at point 11a, the strut may have a circumferential width w that is between about 1.2 and 1.3 times the radial thickness t. In still further aspects, at point 11a, the strut may have a circumferential width w that is between about 1.26 times the radial thickness t. In some aspects of the technology, the struts of housing 11 may have these same proportions (e.g., a circumferential width w being between 1.2 and 1.8 times radial thickness t) at each of points 11b, 11c, and 11d. Likewise, in some aspects of the technology, the struts at points 11a and 11d may each have the same proportion of width w to radial thickness t, while the struts at points 11b and 11c may have proportions that are slightly more square. For example, in some aspects, the struts at points 11a and 11d may have a circumferential width w that is between about 1.2 and 1.8 times the radial thickness t, while the struts at points 11b and 11c may have a circumferential width w between about 1.0 and 1.60 times the radial thickness t. In some aspects, the struts at points 11a and 11d may have a circumferential width w that is between about 1.2 and 1.3 times the radial thickness t, while the struts at points 11b and 11c may have a circumferential width w between about 1.0 and 1.15 times the radial thickness t. In still further aspects, the struts at points 11a and 11d may have a circumferential width w that is about 1.26 times the radial thickness t, while the struts at points 11b and 11c may have a circumferential width w between about 1.09 times the radial thickness t. In this regard, in some aspects of the technology, the radial thickness t may be constant throughout housing 11, while the circumferential width w of the struts may vary along the length of housing 11.

As will be understood, increasing the cross-sectional area of the struts as described herein may lead to the pump housing 11 being substantially stiffer and thus more resistant to kinking and/or plastic deformation, particularly at or around points 11a and 11d, which likewise may reduce the risk of the drive shaft kinking where it passes these same points. In addition, although increasing the circumferential width w of the struts may reduce the area through which blood may flow into and out of housing 11 when the pump is in operation, it has been found that it is possible to increase the circumferential width of the struts in the ranges described herein without substantially increasing flow resistance and hemolysis. Further it has been found that it is possible to increase the circumferential width w of the struts in the ranges described herein without substantially increasing the force required to compress the pump housing and without substantially increasing related implantation forces which in some cases may be correlated with the elastic recoil forces of the compressed pump housing.

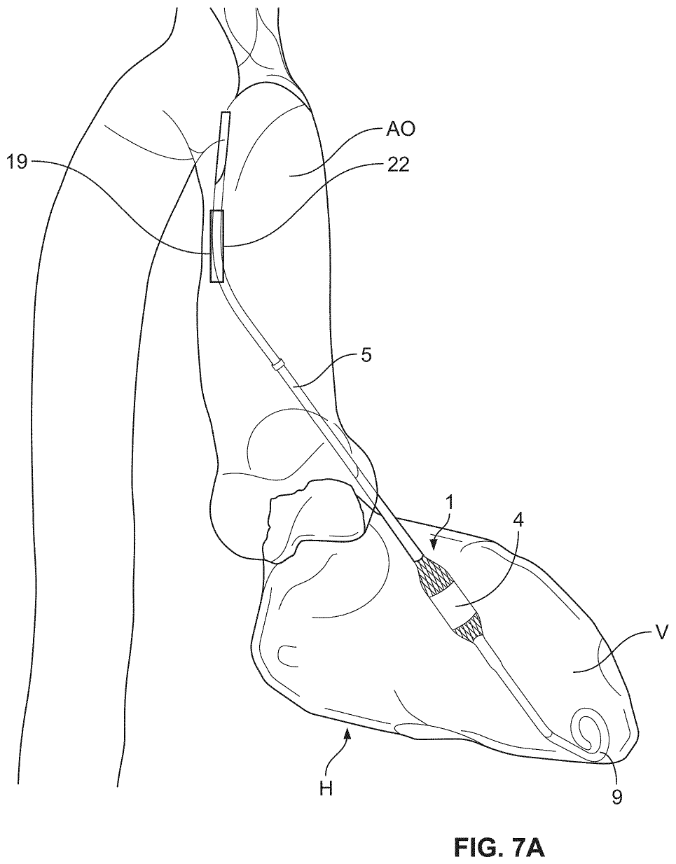

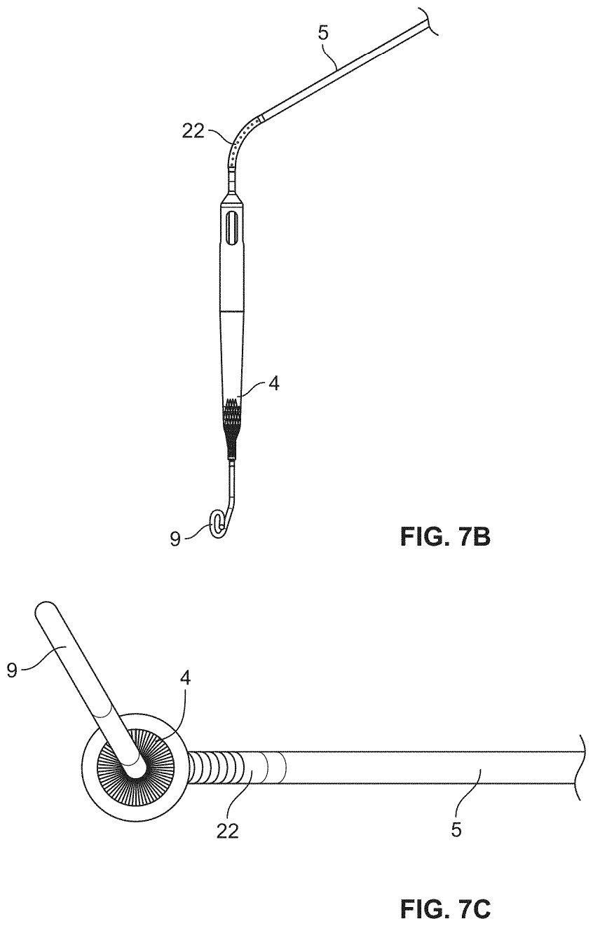

As also described herein, a catheter may be configured to control a position of the intravascular blood pump when deployed in a patient. As described and illustrated in FIG. 7A, for example, a sleeve 22 may be placed over a portion of the catheter joined to a proximal end of the intravascular blood pump 1. In some aspects, the sleeve may be proximal to and adjacent to an outlet of the pump section of the intravascular blood pump. As stated above, the intravascular blood pump may be percutaneously inserted into the heart through the aorta. In such instances, the intravascular blood pump may be generally positioned past the aortic valve in the left ventricle, in order to pull blood from the left ventricle and expel the blood into the aorta. In some embodiments, an atraumatic tip 9 on the far distal end of the intravascular blood pump may contribute to spacing and positioning the pumping section of the blood pump from the heart wall. Consequently, in some instances, the pumping section may be positioned near the walls of the heart or various heart structures, such as the mitral valve. The sleeve described herein may be adapted to better and more precisely control the position of the pumping section of the intravascular blood pump (e.g., allow the positioning the pumping section in the apex of the ventricle (away from the septum and mitral valve)) when inserted into a patient's heart, as will be described in detail below.

FIG. 7A illustrates the intravascular blood pump 1 inserted into the ventricle V of the patient's heart H via the aorta AO. As shown in this view, the catheter 5 may have a distal end that is attached to the proximal end of the pumping section of the intravascular blood pump 1 and a proximal end (not shown) located at the outside of the patient's vasculature and extends therebetween. An impeller (not shown) may be provided in the pumping section to cause the blood flow from the blood flow inlet to the blood flow outlet. The impeller may be driven by a motor that may either be inside the patient and monolithically integrated with the pumping section 4 of the intravascular blood pump 1, or outside the patient.

In some embodiments, the catheter 5 has a lumen (not shown) that extends through the catheter 5. The catheter 5 may have an inner diameter sufficient to provide a space for the drive shaft with a small gap between the drive shaft and the inner wall of the catheter 5, such as, about 1.57 mm (corresponding to a dimension of about 5 French). The catheter 5 may have an outer diameter of about 2.75 to 3.1 mm (corresponding to a dimension of about 8 to 9 French).

Referring again to FIG. 7A, the catheter 5 may be provided with a bend region 19 formed thereon with a sleeve 22 placed thereon. In some embodiments, the bend region 19 may influence the position of the pump section 4 of the intravascular blood pump 1 when inserted into the patient's heart H. Specifically, as the intravascular blood pump 1 is inserted through the aorta AO, the sleeve 22 may follow the plane of the aortic arch, and the bend region 19 may make a contact with the endothelium of the aorta AO, as shown in FIG. 7A, allowing the intravascular blood pump 1 to be supported and allowing the atraumatic tip 9 to be correctly aligned with the aortic valve to position the pump section 4 in the apex of the ventricle V of the heart H. For correctly positioning the atraumatic tip 9 in the apex of the ventricle V of the heart H, the sleeve 22 may need to be placed as close to as possible to the pumping section 4 and be oriented relative to the atraumatic tip 9 such that a valve transfer is easiest by orienting the atraumatic tip 9 over the center of the aortic valve. Such orientation of the atraumatic tip 9 may be from about 110 degrees to 150 degrees relative to the sleeve 22, as shown in FIGS. 7B and 7C (e.g., between 120 and 140 degrees). Said another way the atraumatic tip 9 may be between 110 to 150 degrees, optionally 120 to 140 degrees, and optionally 130 degrees out of plane (plus or minus) with respect to the plane in which the bent sleeve lies flat. This may be readily observed in FIG. 7B where the plane of the sleeve 22 is in page and the plane of the atraumatic tip 9 is out of page and not perpendicular to the plane of the page. FIG. 7C, which is from the perspective of the atraumatic tip 9, reveals that the pigtail extends at an angle from the plane of the sleeve 22. Although the orientation where the atraumatic tip 9 is illustrated as out of plane respect to the plane of the bent sleeve 22 is described above, it is contemplated that the atraumatic tip 9 and the bent sleeve 22 may be arranged in the same plane, with that in plane relationship being preserved by the sleeve 22 when the intravascular blood pump 1 is inserted into the patient and positioned therein.

In some embodiments, as will be appreciated in view of the above, the atraumatic tip 9 also may be arranged out of the plane with respect to the catheter bend. The atraumatic tip 9 also may be arranged in the plane of the catheter bend in other embodiments.

The relaxed state of the bend region 19 defined on the catheter 5 is maintained using the deformable sleeve 22 placed thereon as the intravascular blood pump 1 is inserted into the aorta AO. The relaxed state preserves both the bend of the catheter 5 in its plane and the out of plane relationship between the sleeve 22 and the atraumatic tip 9. The deformable sleeve 22 is designed and configured to be placed in or on the bend region 19 of the catheter 5 during operation of the intravascular blood pump 1 in order to support the catheter 5 during the entire surgical procedure and during operation of the intravascular blood pump 1. In this regard, the deformable sleeve 22 may be placed over the bend region 19 of the catheter. The deformable sleeve also may be embedded into the wall of the catheter 5 in the bend region 19 (i.e., in the interior of the catheter). In some embodiments, the sleeve may be placed over the exterior of the catheter. In some embodiments, a polymeric tube may be attached to the catheter, with the sleeve being placed around the exterior of the polymeric tube and catheter.

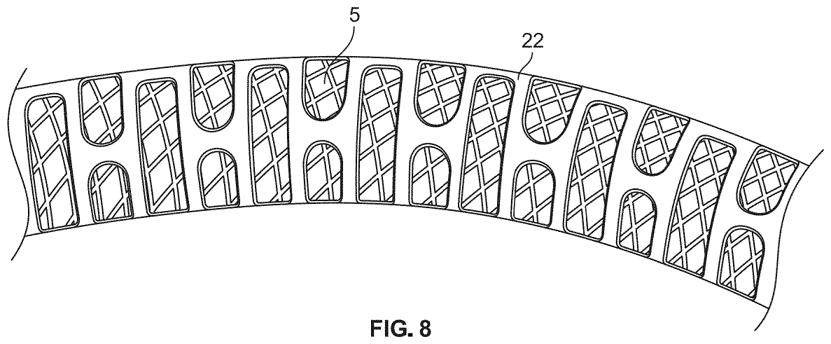

Referring to FIG. 8, in the embodiment where the sleeve 22 is coupled to the catheter 5 (e.g., attached to the exterior of the catheter), the inner diameter of the sleeve 22 may be slightly larger than the outer dimeter of the catheter 5, allowing the sleeve 22 to be moved axially along the length of the catheter 5 to be placed in the bend region 19 with the application of force in the axial direction. Once the sleeve 22 is at the bend region 19, the sleeve 22 may be firmly affixed to the catheter 5 with a suitable means for fixation such as gluing, sonic welding, etc. One skilled in the art is aware of suitable means for fastening the sleeve to the catheter. In other embodiments, the sleeve 22 may be embedded in the catheter 5 as described below. In some embodiments, sleeve 22 may be embedded in a polymeric material (e.g., polyurethane) used to form the catheter 5. As will be appreciated, catheter construction is well known and, thus, not described in detail herein. In one example, the catheter 5 may be formed of polyurethane extruded on a mandrel. In one example, a braided metal (e.g., stainless steel, nitinol, etc.) may be pulled over the extruded polyurethane and melted into the tube. The sleeve 22 is then placed over this structure. More polymer (e.g., polyurethane) may then be formed over this structure. In some aspects of the technology, the sleeve 22 may be embedded in (or covered by) a material that is different than that of adjacent sections of the catheter 5. For example, catheter 5 may include a polymer sleeve that is predominantly made from a harder and stiffer polymer (e.g., one with a hardness between 95A and 72D, such as Carbothane 72D), but which includes an intermediate section of a softer polymer (e.g., one with a hardness between 55D and 65D) that partially or fully overlaps a sleeve 22. In some cases, sleeve 22 may be sandwiched between an inner layer and an outer layer of polymer, in which both the inner and outer layers are predominantly made from a harder polymer with an intermediate section. In some aspects, the intermediate section of the inner layer may be staggered with respect to the sleeve 22, and the sleeve 22 may further be staggered with respect to the intermediate section of the outer layer, such that the overall stiffness of the assembly changes more gradually. Likewise, in some aspects, the intermediate section of the inner layer may be a different length than the intermediate section of the outer layer, such that a sleeve 22 may be fully overlapped (or underlapped) by the intermediate section of one layer, while extending beyond one or both ends of the other layer. As will be appreciated, in some aspects of the technology, the catheter 5 may employ additional sections beyond those just described, such as a section on one or both sides of the intermediate section having an intermediate hardness (e.g., 65D-72D). The catheter 5 may also employ additional layers of polymer in one or more of these sections.

The sleeve 22 may have a preformed bend that may be straightened when placed on the catheter under construction. In one example, the sleeve 22 is bent by annealing the sleeve in a bent configuration. Other heat treatments for forming the sleeve are contemplated. In one example, the sleeve 22 may be heated on a mandrel to introduce the bend in the sleeve 22. The sleeve 22 will have a preformed bend that may be straightened when placed on the catheter under construction. The sleeve 22 will relax back to its preformed bend after fabrication.

In some embodiments, the sleeve 22 may allow the catheter 5 to maintain the predefined bend region 19 such that the placement of the pump section 4 of the intravascular blood pump 1 in a desired position may be achieved when inserted into a patient's heart. Specifically, as stated above, the predefined bend region 19 on the catheter 5 with the sleeve 22 thereon may contribute to the desired alignment of the atraumatic tip 9 with the aortic valve during insertion and also contributes to positioning the atraumatic tip 9 in the apex of the ventricle V. The sleeve 22 also stabilizes and prevents the pump section 4 from rotating as it travels through the aortic arch. The sleeve 22 also may avoid the need to torque the catheter 5 further to properly position the pump section 4 in the heart after it has been introduced therein as such torquing may cause tissue damage to the patient's vasculature or heart.

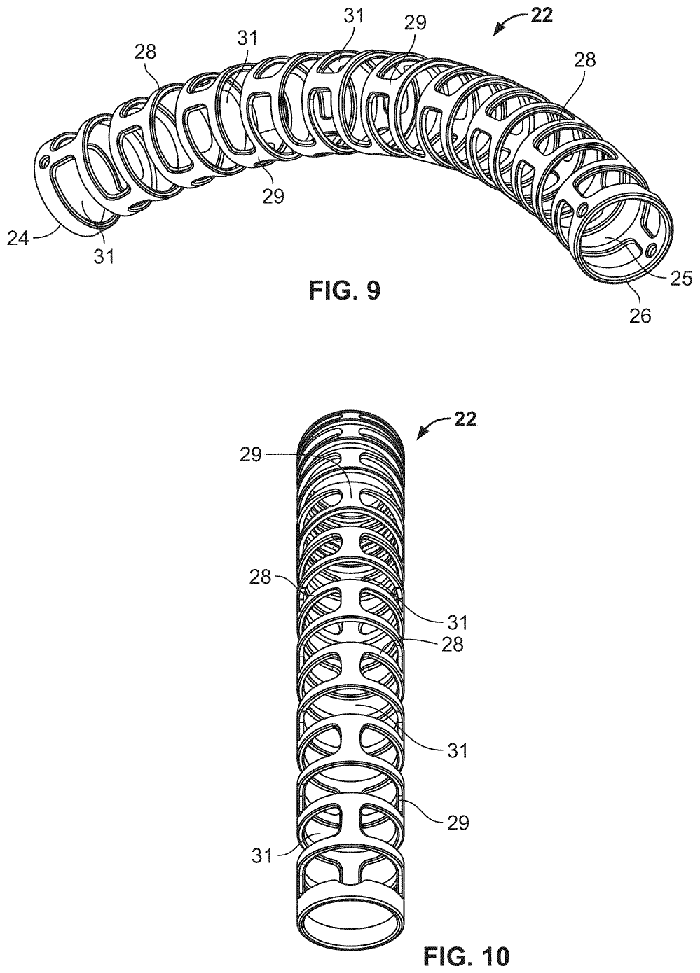

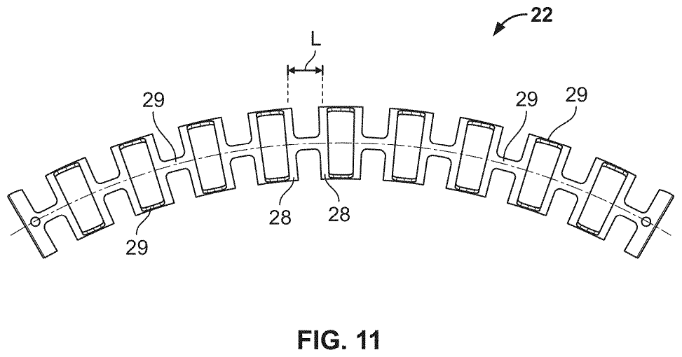

Referring to FIGS. 9-11, in one embodiment, the illustrated sleeve 22 is configured to be placed and disposed over, in, or on the bend region 19 of the catheter 5. FIG. 9 is a perspective view of the sleeve 22 where the plane of the bend is observed. FIG. 10 is a top perspective view where the bend of the sleeve 22 occurs into the page. FIG. 11 is a top view of the sleeve 22 with the bend observed in the plane of the page. The sleeve 22 may be annular and extend between a first open end 24 and a second open end 26 (see FIG. 9). The sleeve 22 may define a partially open lumen 25 that extends between the first open end 24 of the sleeve 22 and the second open end 26 of the sleeve 22. The lumen 25 may be sized such that the sleeve 22 may be slid along the catheter 5 (at some phase of catheter fabrication) in the axial direction and disposed in the designated bend region 19 of the catheter 5. In other embodiments, the lumen 25 is sized so that it may be embedded in outer layer of the catheter 5. As noted herein, the designated bend region 19 may be proximal to the pumping section 4. In one embodiment, the bend region 19 may be proximal to and adjacent to the pumping section 4. In other embodiments, the bend region 19 may be proximal to, but not adjacent to, the pumping section 4.

The sleeve 22 illustrated in FIGS. 9-11 may include a series of spaced apart annular rings 28 wherein adjacent rings 28 are joined by at least a pair of connectors 29. In some embodiments, the connectors 29 are not aligned, but instead may be offset from ring pair to ring pair. As such, a plurality of openings 31 may be formed on the sleeve 22 between each ring pair and arranged in an alternating repeating fashion to form a particular pattern. Specifically, the plurality of openings 31 are formed in radially matched pairs which define a semicircle of 180 degrees about the circumference of the sleeve 22. Each of the openings 31 may extend approximately halfway around the circumference of the sleeve 22 and is separated by the connectors 29. As noted above, the pairs of openings 31 may be offset circumferentially from ring pair to ring pair on the sleeve 22 to form the pattern, as shown in FIGS. 9 and 10, with the pairs of openings 31 being parallel to but offset from one another in an alternating fashion. Each opening 31, at the connector terminus of the opening, has non-uniform radii. For example, the radius at each corner of the opening 31 (where the connector and ring are connected) is different from the radius along the connector 29 and the terminus of the opening 31 in the ring 28.

The non-uniform radii of the openings 31 may be readily observed in FIG. 11. In some embodiments, there are two connectors per ring pair. The connectors may be 90 degrees offset from ring pair to ring pair such that only the top connector 29 is visible for one set of ring pairs, but two connectors 29 are visible for the other ring pair. As will be appreciated, in other embodiments, one or more connectors may be used between ring pairs. As will be further appreciated, the same number of connectors may be used between all ring pairs, although the number of connectors may vary between ring pairs.

Viewing the space “L” between the two rings, it may be seen that there is a tighter, smaller radius in the corner of the transition from the connector 29 with the ring 28 than there is between those two corners. That is what is meant by the reference to a non-uniform radius for the openings 31. The plurality of annular rings 28 may be spaced apart in a uniform length L when in the straight configuration. FIG. 11 illustrates a longitudinal length L being measured between a longitudinal center point of adjacent rings 28. In some cases, the longitudinal length L may be generally constant between all adjacent rings 28 along the length of the sleeve 22 when the sleeve 22 is in a straight position.

As illustrated in FIG. 10, each of the plurality of openings 31 may be about equal in size (e.g., length, width, and area) such that the plurality of openings 31 are also substantially identical when the sleeve 22 is in a straight position. The length of the sleeve 22 may be dimensioned to extend the length of the predefined bend region 19 on the catheter 5. As illustrated in FIG. 11, bending the sleeve 22 will introduce deformation of the spacing at the apex of the bend, with the spacing of L getting larger on the exterior of the bend and the spacing L getting smaller on the interior of the bend. The configuration and design of the plurality of rings 28 and connectors 29 may be configured to allow the sleeve 22 to be bent in different directions.

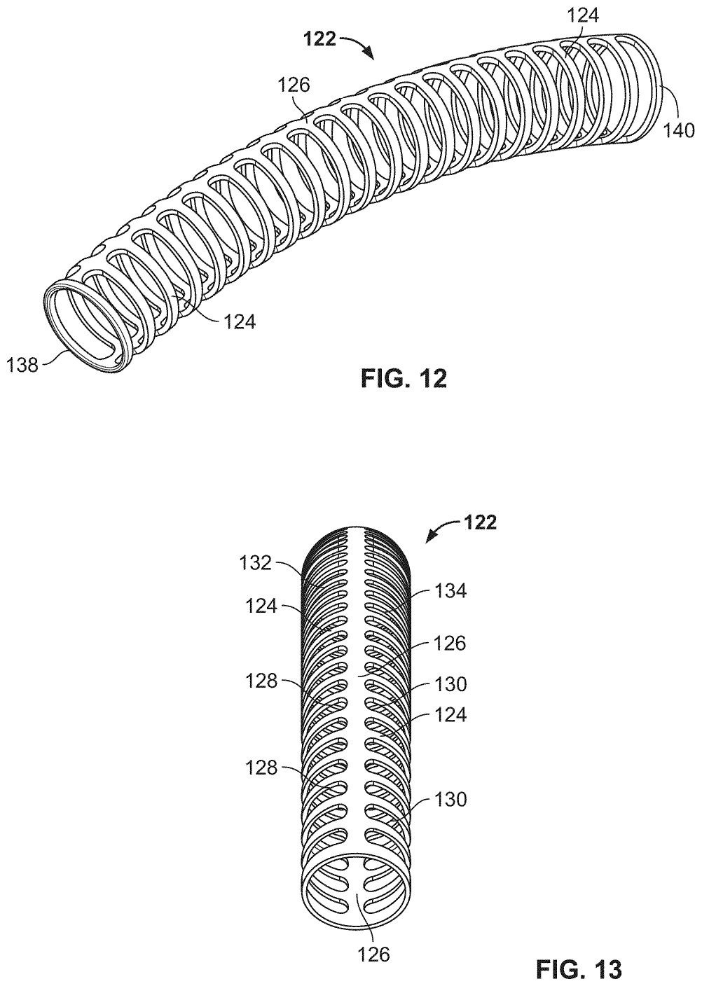

Referring to FIGS. 12-14, in a second embodiment, the sleeve 122 structure may include a series of spaced apart annular rings 124 joined by two axial spines 126 that extend the length of the sleeve (i.e., there is no offset). As such the sleeve 122 includes a plurality of first openings 128 and a plurality of second openings 130 on either side of the axial spines 126. That is, the sleeve 22 is symmetrical. As illustrated, each of the first and second openings 128, 130 is defined on the sleeve 122 and extends about one-half way around the circumference of the sleeve 122, but this arrangement is merely illustrative. Configurations with one spine or more than two spines 126 are also contemplated. The spines 126, as illustrated, may be spaced approximately 180 degrees from each other. However, in the embodiments with two spines, the angular spacing is a matter of design choice with angular separations of 45 degrees to 180 degrees being contemplated. As illustrated, the plurality of first openings 128 may be parallel to one another, and the plurality of second openings 130 also may be parallel to one another, as shown in FIG. 13.

As shown in FIG. 13, for example, each of the plurality of first openings 128 may be defined on a first, e.g., left portion 132 of the sleeve 122, while each of the plurality of second openings 130 may be defined on a second, e.g., right portion 134 of the sleeve 122. The plurality of openings 128, 130 may be positioned laterally and be evenly spaced apart along a length of the sleeve (or a longitudinal axis of sleeve) 122, forming the plurality of rings 124 between the plurality of openings 128, 130, as shown in FIGS. 12 and 13.

As illustrated, each of the plurality of openings 128, 130 may be approximately equal in size (e.g., length, width, and area) such that the plurality of openings 128, 130 also may be substantially identical when the sleeve 122 is in a straight position. The length of the sleeve 122 may be dimensioned to extend the length of the predefined bend region 19 on the catheter 5.

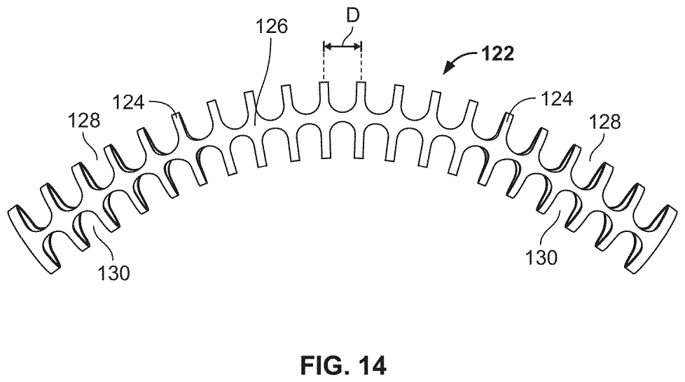

As shown in FIG. 14, each of the plurality of rings 124 may be interconnected with a pair of spines (or support members) 126. Each spine 126 may be substantially straight in configuration and substantially parallel to the longitudinal axis of the sleeve 122. The spines 126 may extend along the length of the sleeve 122, such as between a first open end 138 of the sleeve 122 and a second open end 140 of the sleeve 122 and are positioned diametrically opposed from each other.

The plurality of annular rings 124 may be, as illustrated, spaced apart a uniform length distance D when in the straight configuration. FIG. 14 illustrates a longitudinal length distance D being measured between a longitudinal center point of adjacent rings 124. Typically, the longitudinal length distance D is generally constant between all adjacent rings 124 along the length of the sleeve 122 when the sleeve 122 is in a straight position. However, it will be appreciated that the longitudinal length distance D may vary between adjacent rings in other embodiments. In some embodiments, while the plurality of openings 128, 130 and the plurality of rings 124 allow the sleeve 122 to be bent to the left and to the right, the spines 126 may define the arc of the curve of the sleeve 122. As noted above, in the bent position, the distance D might be slightly greater on the outside of the curve compared with the distance D on the inside of the curve. A catheter may be formed using the sleeve illustrated in FIGS. 12-14 in the manner described above.

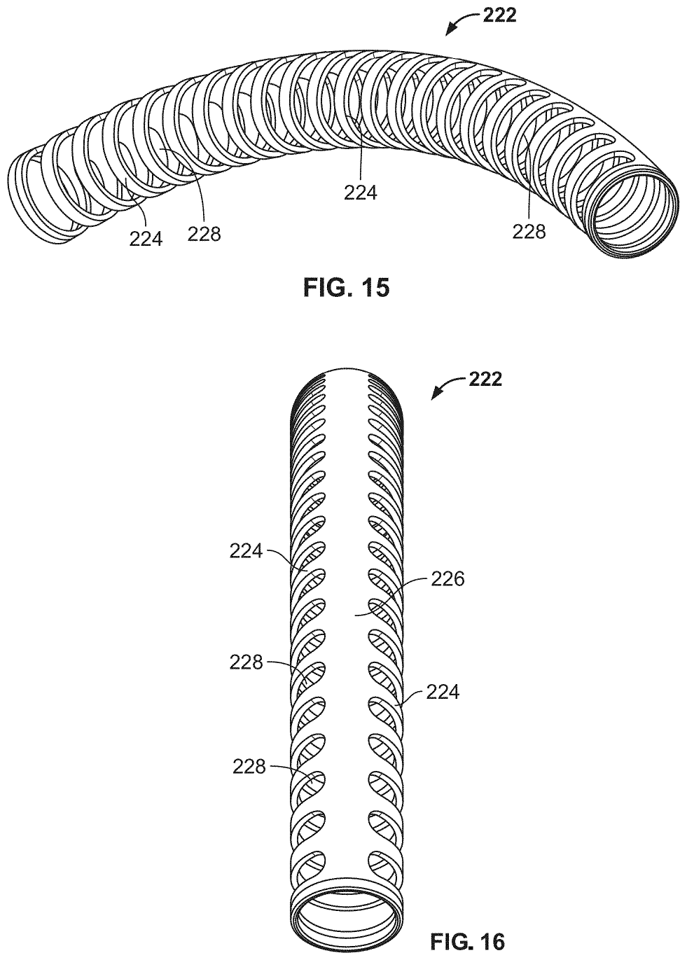

FIGS. 15 and 16 illustrate a different sleeve where the bend may be observed in the plane of the page in FIG. 15 and extending into the page in FIG. 16 (both FIGS. 15 and 16 are perspective top views). Referring to FIGS. 15 and 16, in a third embodiment, the sleeve 222 may include a series of spaced apart annular rings 224 joined by a single axial spine 226. A plurality of openings 228 may be defined between each annular rings 224 throughout the length of the sleeve 222 but for the spine 226 that traverses each opening 228 between each annular ring 224. A catheter may be formed using the sleeve illustrated in FIGS. 15 and 16 in the manner described above.

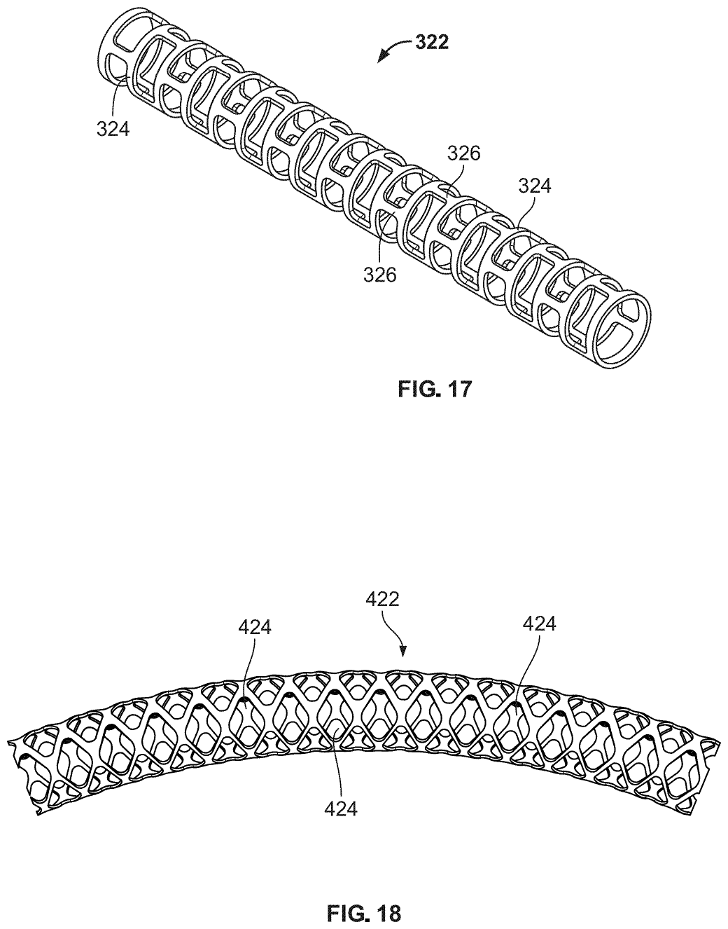

Referring to FIG. 17, in a fourth embodiment, the sleeve 322 (illustrated as being unbent) may include a series of spaced apart annular rings 324 connected by a plurality of connectors 326 disposed between each of the annular rings 324. As with other embodiments described herein, the connectors 326 may be circumferentially offset from each other from ring pair to ring pair, causing an offset in the openings between the pairs of rings 324. The sleeve 322 may include an alternate embodiment of the embodiment shown in FIGS. 9-11, as will be appreciated. In some embodiments, a catheter may be formed using the sleeve illustrated in FIG. 17 in the manner described above.

Referring to FIG. 18, in a fifth embodiment, the sleeve 422 (illustrated as bent) may include a plurality of diamond-shaped apertures 424 formed by helical ribs that traverse the length of the sleeve 422. The helical patterns may overlap and intersect to define the pattern of apertures 424. The plurality of apertures 424 may be formed on the sleeve 422 to enable bending of the sleeve 422 while still providing axial stiffness and maintaining axial strength. A catheter may be formed using the sleeve illustrated in FIG. 18 in the manner described above.

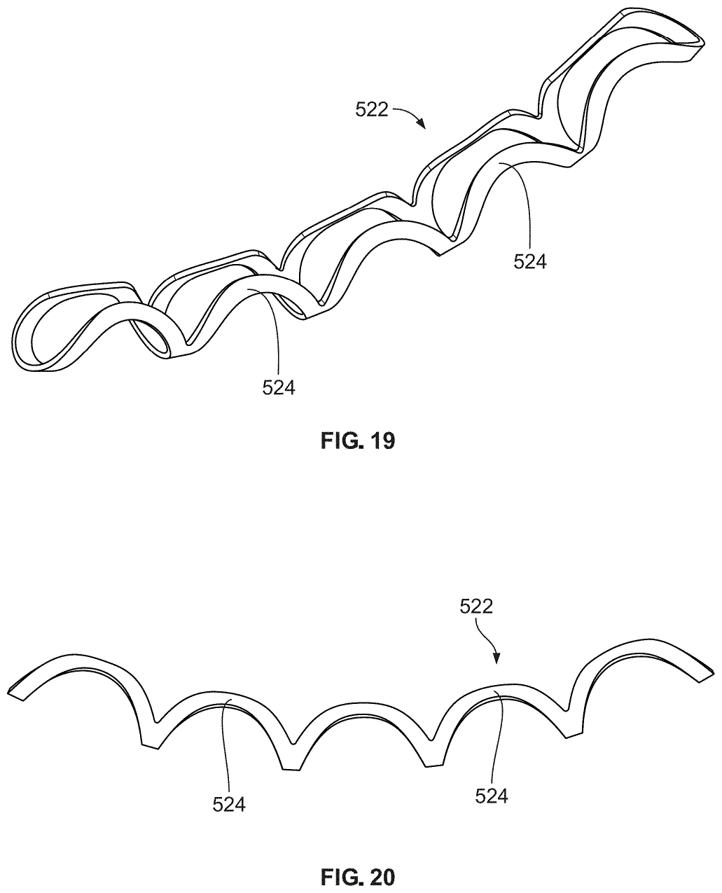

Referring to FIGS. 19 and 20, in a sixth embodiment, the sleeve 522, also illustrated as bent, may include a series of open cradle structures 524 (each structure having open top and open bottom) that are joined together. The cradle structure 524 of the sleeve 522 may not surround the catheter in such embodiments, but instead may be disposed on only one side of the catheter. As such, the open side of the cradle structures 524 may curve toward each other to snugly fit over the catheter. As shown in FIG. 20, each structure 524 may have an arch like configuration that allows the cradle structures to partially surround the catheter. A catheter may be formed using the sleeve illustrated in FIGS. 19 and 20 in the manner described above.

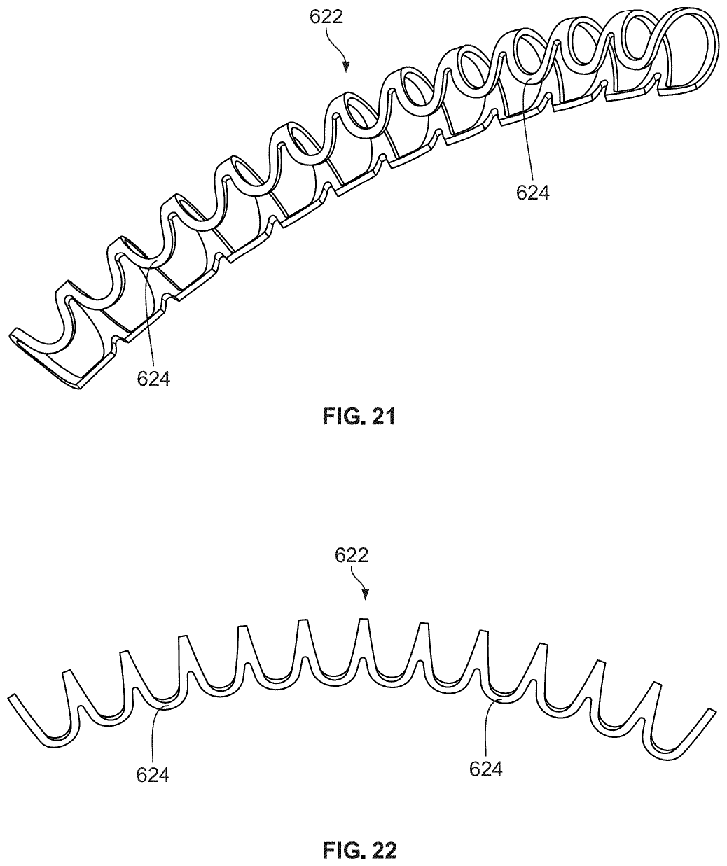

Referring to FIGS. 21 and 22, in a seventh embodiment, the sleeve 622, illustrated as bent, may include a series of more tightly spaced cradle structures 624 (each cradle structure having open top and open bottom) that are joined together. As shown in FIG. 22, each structure 624 may include an arch that is more U-shaped in the side view than the arches in the cradle structures of FIGS. 19 and 20. Inn some embodiments, a catheter may be formed using the sleeve illustrated in FIGS. 21 and 22 in the manner described above.

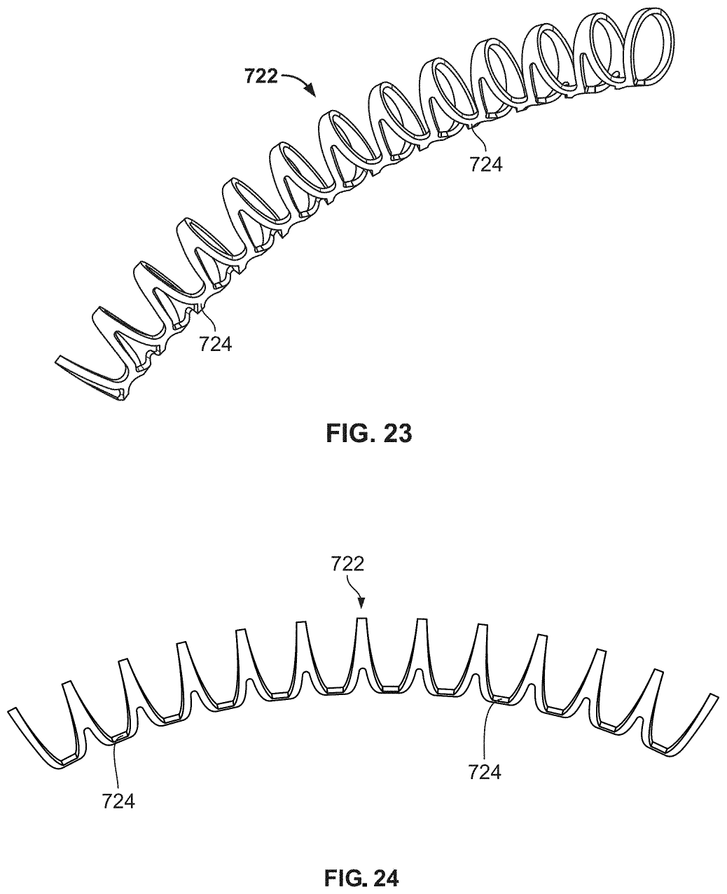

Referring to FIGS. 23 and 24, in an eighth embodiment, the sleeve 722, illustrated as bent, may include a series of annular ring structures 724 (each structure having an open top) that are joined together with U-shaped connectors. In such embodiments, the connectors may be all disposed on the same side of the sleeve 722. In some embodiments, a catheter may be formed using the sleeve illustrated in FIGS. 23 and 24 in the manner described above.

The sleeve 22, 122, 222, 322, 422, 522, 622, 722 is made of one or more materials having suitable properties for a desired application, including strength, weight, rigidity, etc. The sleeve may have flexible areas to allow for the sleeve to be bent in a predetermined configuration, or have malleable areas to allow the user to adjust the support structure to individual needs of the patient.

The sleeve 22, 122, 222, 322, 422, 522, 622, 722 may be made of conventional materials that are biologically compatible (e.g., stainless steel). Optionally, the sleeve may comprise or be made of a shape-memory material (e.g., a shape-memory alloy, in particular Nitinol). The sleeves described herein may be formed in any conventional manner (e.g., laser cutting). Because of this material, the sleeve may allow the catheter to be bent, i.e., elastically deformed, with a bending radius of between 15 mm and 90 mm, or between 18 mm and 60 mm, or between 21 mm and 31 mm. The bending radius is measured with respect to a central axis of the catheter. The desired bending stiffness characteristics result mainly from the superelastic properties of the Nitinol.

In some embodiments, one or more sleeves may be used to shape the catheter at a desired location. As will be appreciated, other methods may be used to effectuate the desired shape (e.g., bend) of a portion of the catheter. For example, a nitinol wire without a sleeve may be used. In other embodiments, the catheter could be pre-bent. In still other embodiments, Kevlar fibers may be used to maintain the desired shape (e.g., bend).

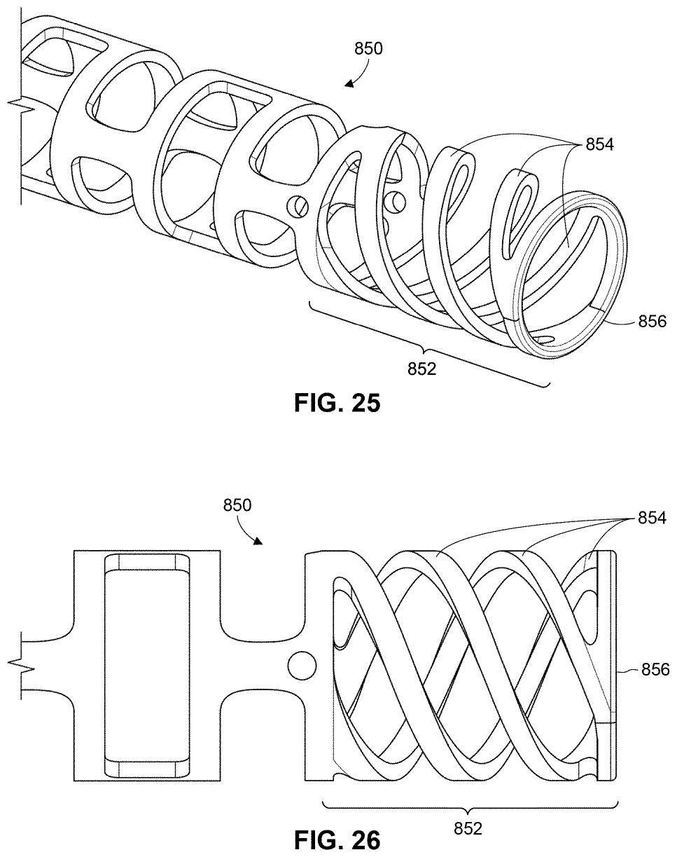

Turning now to FIG. 25-28, in some embodiments, a sleeve (e.g., sleeve 850 of FIGS. 25-28, and/or any one of sleeves 22, 122, 222, 322, 422, 522, 622, 722 of FIGS. 7A-24) may be formed with a strain relief section on one or both of the proximal and distal ends of the sleeve. In such embodiments, the strain relief sections may help to reduce strain peaks in the material of catheter 5 where it is coupled to an end of the sleeve. Such strain relief sections may be any suitable length compared to the total length of the sleeve. For example, in some embodiments, a sleeve may be between 15 and 30 mm, with the strain relief section being 3-5 mm thereof.

In some embodiments, the strain relief sections may allow the sleeve, and in turn the catheter 5, to be more flexible. The stiffness of the such strain relief sections may be configured in a number of ways, such as by selecting a particular length, maintaining a particular ratio between its length and its diameter (e.g., setting its length to be at least 0.5 times its diameter, at least 1 times its diameter, at least 1.5 times its diameter, etc.), choosing how many struts it employs, choosing the thickness of such struts, choosing the pitch of the struts (where spiral struts are employed), and/or by embedding or covering the struts with a material of a particular hardness or flexibility.

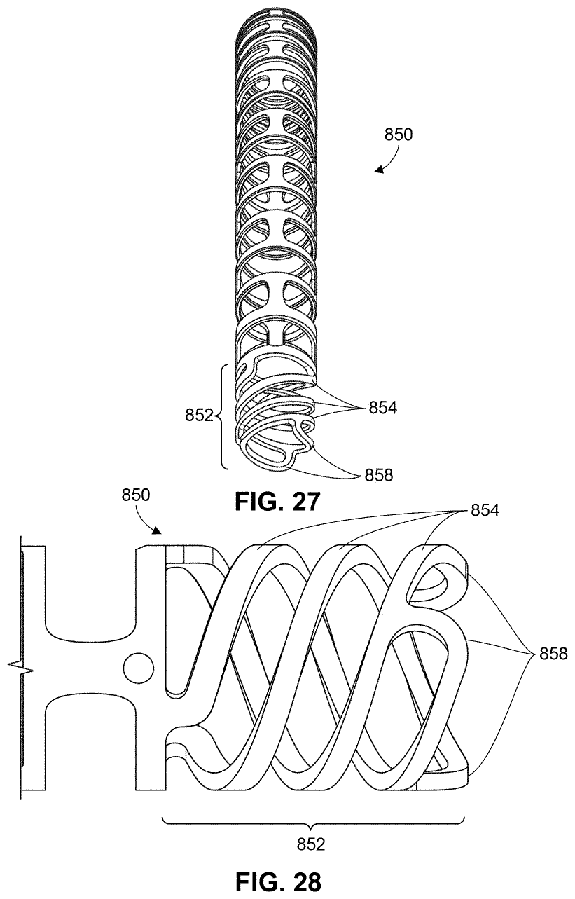

In addition, in some embodiments, the strain relief sections may be configured to have a stiffness that varies over a length of strain relief section. In some embodiments, the stiffness of the strain relief section may be configured to continuously reduce from the end of the main section of the sleeve (e.g., with one or more annular ring sections) to the end of the strain relief section. In some embodiments, this may be achieved by using one or more spiral struts in the strain relief section, where the widths of the struts change over the length of the strain relief section. In that regard, in the examples of FIGS. 25 and 26, each of the three struts 854 are shown continuously reducing in thickness as they approach end 856. In some embodiments, the stiffness of the strain relief section may be varied over the length of the strain relief section by continuously changing the pitch of one or more spirally shaped struts (e.g., struts 854). In still other embodiments, the stiffness at one end of a strain relief section may be further adjusted based on how each spiral strut terminates. For example, as shown in FIGS. 27 and 28, each spiral strut 854 may end in loops 858 connecting to another strut, which may lead to a lower stiffness at that end than by having each strut terminate in a full ring, as shown at end 856 of FIGS. 25 and 26. Further, in some embodiments, the stiffness of the strain relief section may be varied over the length of the strain relief section by changing the material of catheter 5 over a length of the strain relief section. For example, in some embodiments, a harder and/or stiffer type of polymer may be used to cover the sleeve at one end of the strain relief section than at the other end of the strain relief section. Likewise, in some embodiments, a thicker layer of polymer may be used to cover the sleeve at one end of the strain relief section than at the other end of the strain relief section.

The strain relief sections 852 of FIGS. 25-28 may be formed in any suitable way, including using any of the methods described above with respect to sleeves 22, 122, 222, 322, 422, 522, 622, 722 of FIGS. 7A-24. Thus, for example, in some embodiments, the strain relief sections 852 may be formed via laser-cutting a sheet or tube of a suitable raw material (e.g., a shape-memory alloy such as Nitinol) in a straight configuration. The sheet or tube may then be processed, such as via a heat treatment, to achieve a desired heat treatment.

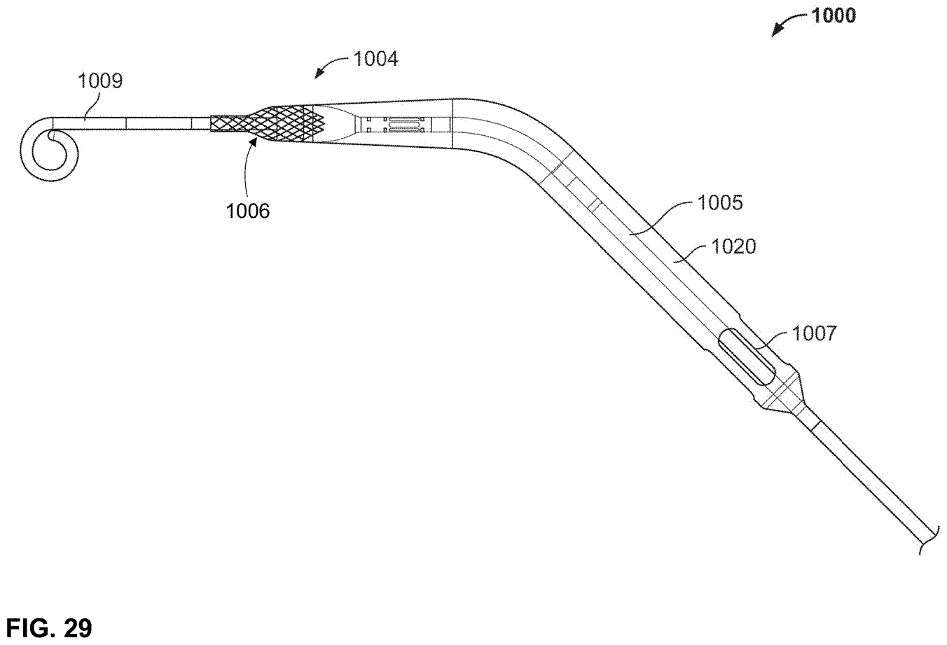

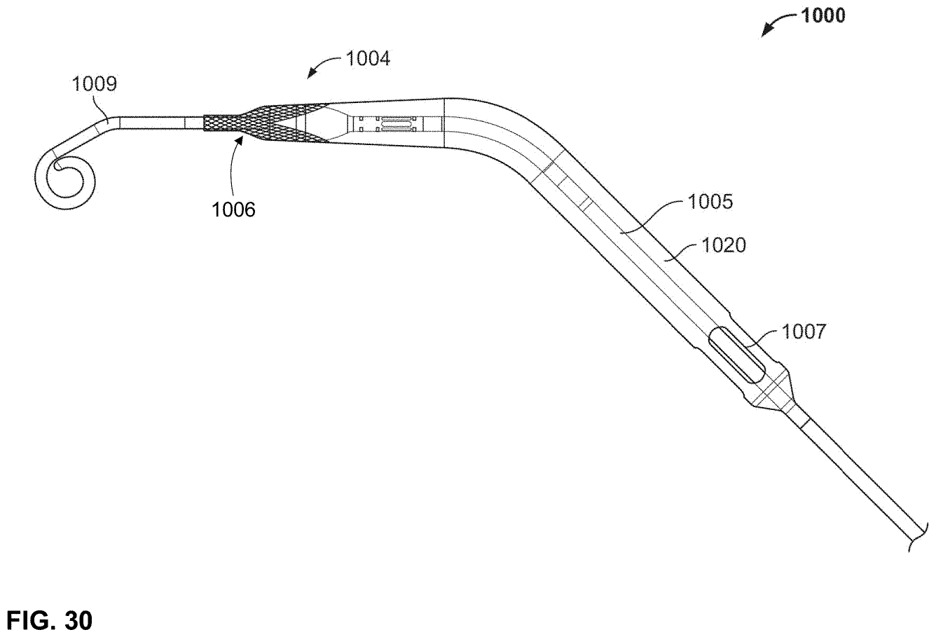

FIGS. 29 and 30 illustrate additional examples of an intravascular pump 1000 according to other embodiments of the present design. As shown in these views, and similar to other pumps described herein, pump 1000 may include a catheter 1005 and a pump section 1004 mounted at a distal region of the catheter 1005. The pump section 1004 may include a rotor (not shown) that may allow blood to flow from a blood flow inlet 1006 to a blood flow outlet 1007. As shown in FIGS. 29 and 30, the pump also may include a flexible atraumatic tip 1009, such as a pigtail, which may be configured to facilitate placement of the pump in the patient's vascular system. In some embodiments, as shown in FIG. 29, the pigtail may include a straight configuration. Likewise, in some embodiments, as shown in FIG. 30, the pigtail may include a bent configuration.

As shown in FIGS. 29 and 30, the pump 1000 may include downstream tubing 1020 through which the catheter 1005 is disposed. As with the above, the downstream tubing 1020 may be made of a flexible material or materials such that it may be compressed by the aortic valve as the patient's heart is pumping. For example, the downstream tubing 1020 may include a balloon. Likewise in some embodiments, the tubing 1020 may be configured to expand as a result of a blood flow generated by the rotor during rotation.

The downstream tubing and catheter may have any suitable shape and configuration. For example, as shown in FIG. 2, the downstream tubing 20 and the catheter 5 may include a straight configuration. In other embodiments, as shown in FIGS. 29 and 30, the catheter 1005 may include a bent configuration. In such embodiments, the downstream tubing 1020 also may include a bent configuration, with the bent catheter 1005 extending through the bent downstream tubing 1020. As will be appreciated, in some embodiments, the catheter 1005 also may include one or more straight regions (e.g., downstream or upstream of the bend), with the downstream tubing 1020 also having corresponding straight regions.

In embodiments in which the catheter 1005 and downstream tubing 1020 are both bent, the bend angle (e.g., radius) of the catheter and the bend angle (e.g., radius) of the downstream tubing may be the same (e.g., 45°±10°). In other embodiments, the bend angle of the catheter and the bend angle of the downstream tubing may differ. For example, the bend angle of the catheter may include 45°±10° while the bend angle of the downstream tubing may include 30°±10°. In such embodiments, the difference in the bend angles may account for the difference in materials between the catheter and the tubing and the way in which the catheter and tubing behave in the patient's body.

In other embodiments, the difference in bend angles may be used to account for activity of the pump during insertion. For example, to insert the pump in the patient, the pump may first be retracted into an introducer sheath, which is thereafter advanced into the patient's vasculature. In such embodiments, both the catheter and downstream tubing may remain in a straight configuration in the introducer sheath during delivery. When the pump is thereafter deployed from the introducer and into the patient, the catheter and the downstream tubing may not rebound to the same bend angles. For example, in some embodiments, after deployment, the catheter may not return to the 45°±10° bend angle. Instead, once deployed from the introducer sheath, the catheter may have a different bend angle. In some embodiments, the initial bend angles of the catheter and of the downstream tubing may be configured such that they are different when formed, but will be similar after deployment into the body (and from the introducer sheath).

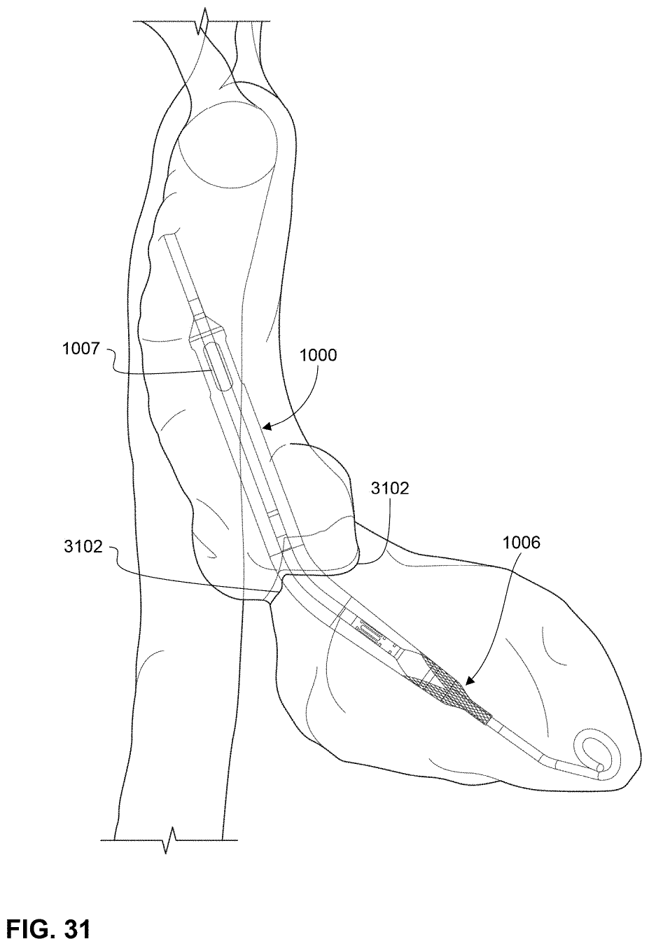

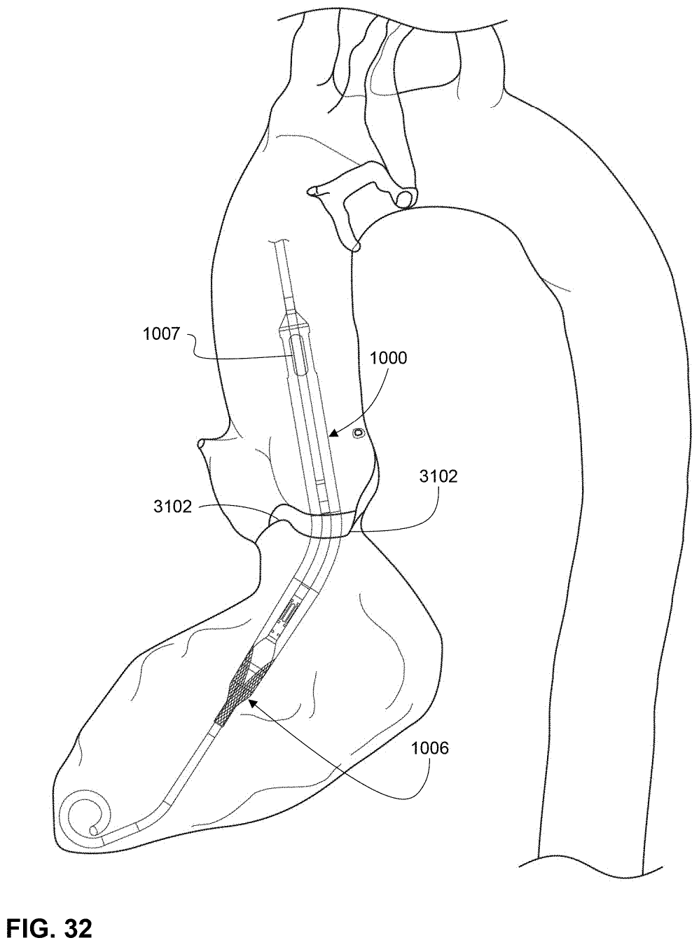

The length of the downstream tubing 1020 between the blood flow inlet 1006 and the blood flow outflow 1007 may be longer in some embodiments than in others (c.f., the amount of downstream tubing 20 between blood flow inlet 6 and blood flow outlet 7 in FIG. 2 with the amount of downstream tubing 1020 between blood flow inlet 1006 and blood flow outlet 1007 in FIGS. 29 and 30). As will be appreciated in view of the pumps shown in FIGS. 31 and 32, a longer region of downstream tubing 1020 between the blood flow inlet 1006 and the blood flow outflow 1007 may make it easier to ensure that the pump 1000 is placed properly across the valve 3102 when the pump is in the patient, and/or that the pump 1000 will be less likely to be inadvertently shifted out of its intended position (e.g., shifted such that the blood flow inlet 1006 and the blood flow outlet 1007 both end up on the same side of the valve 3102, shifted such that the blood flow inlet 1006 or the blood flow outlet 1007 becomes fully or partially covered by valve 3102, etc.). As will also be appreciated in view of the pumps shown in FIGS. 31 and 32, placing a bend in the catheter 1005 and/or the downstream tubing 1020 may likewise make it easier to ensure that the pump 1000 will rest stably across the valve 3102 when the pump is in the patient, and/or that the pump 1000 will be less likely to shift out of its intended position. For example, in some embodiments, the length between of downstream tubing (e.g., downstream tubing 20, 1020) between the blood flow inlet (e.g., blood flow inlet 6, 1006) and the blood flow outlet (e.g., blood flow outlets 7, 1007) may be greater than 20 mm, greater than 30 mm, greater than 40 mm, greater than 50 mm, greater than 60 mm, greater than 70 mm, or even greater than 80 mm.

The term “about” as used herein, is used consistent with how one of ordinary skill in the art would interpret the term relative to the dimension or quantity or value described. That is, the term “about” indicates that there may be some variability in the expressed value, but wherein the objectives of the expressed value may still be met. Absent express statements elsewhere, +/−10% of the expressed value is encompassed by the term “about.”

From the foregoing and with reference to the various figure drawings, those skilled in the art will appreciate that certain modifications may also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

### EXEMPLARY IMPLEMENTATIONS

As already described, the intravascular blood pump described herein may be implemented in various ways. In that regard, the foregoing disclosure is intended to include, but not be limited to, the systems, methods, and combinations and subcombinations thereof that are set forth in the following categories of exemplary implementations.

Category A:

A0. An intravascular blood pump, comprising:
a catheter;
a housing in which a rotor is housed, the housing being attached to a distal end of the catheter; and
a drive shaft extending through the catheter and connected to the rotor, at least a portion of the drive shaft being flexible, the drive shaft comprising an outer layer of wound or braided wires, an inner layer of wound or braided wires, and a reinforcement element arranged within at least the outer layer of wound or braided wires,
wherein the drive shaft is rotatably supported in a proximal bearing located proximal of the rotor and a distal bearing located distal of the rotor,
wherein the reinforcement element extends from at least a point within the proximal bearing to a point within the distal bearing wherein a catheter having a distal end and a predefined bend region positioned proximal to the distal end;
wherein the catheter comprises a sleeve configured to control a position of the pumping device in a patient's heart, the sleeve comprising:
a plurality of annular rings;
at least one connector, the at least one connectors disposed between each annular ring for connecting each of the plurality of annular rings, the at least one connectors being offset from adjacent connectors; and
a plurality of openings formed between each ring,
wherein the sleeve is configured to be monolithically integrated with or placed over the predefined bend region of the catheter and thereby provide a predefined resilient bend in the catheter at the predefined bend region.

A1. An intravascular blood pump, comprising:
a catheter;
a housing in which a rotor is housed, the housing being attached to a distal end of the catheter; and
a drive shaft extending through the catheter and connected to the rotor, at least a portion of the drive shaft being flexible, the drive shaft comprising an outer layer of wound or braided wires, an inner layer of wound or braided wires, and a reinforcement element arranged within at least the outer layer of wound or braided wires,
wherein the drive shaft is rotatably supported in a proximal bearing located proximal of the rotor and a distal bearing located distal of the rotor,
wherein the reinforcement element extends from at least a point within the proximal bearing to a point within the distal bearing wherein a catheter having a distal end and a predefined bend region positioned proximal to the distal end;
wherein the catheter comprises a sleeve configured to control a position of the pumping device in a patient's heart, the sleeve comprising:
a plurality of annular rings;
at least two connectors, the at least two connectors disposed between each annular ring for connecting each of the plurality of annular rings, the at least two connectors being offset from adjacent connectors; and
a plurality of openings formed between each ring,
wherein the sleeve is configured to be monolithically integrated with or placed over the predefined bend region of the catheter and thereby provide a predefined resilient bend in the catheter at the predefined bend region.

A2. The intravascular blood pump of A1, wherein the reinforcement element extends from a point proximal to the proximal bearing to a point within the distal bearing.

A3. The intravascular blood pump of any of A1-A2, wherein the proximal bearing comprises a bearing sleeve attached to the drive shaft and an outer bearing ring attached to the housing, the bearing sleeve being configured to rotate within the outer bearing ring.

A4. The intravascular blood pump of A3, further comprising a restriction element attached to the housing and located proximal of the proximal bearing and configured to prevent the bearing sleeve from becoming dislodged from the outer bearing ring.

A5. The intravascular blood pump of any of A1-A4, wherein the reinforcement element comprises a stepped proximal end with a portion of reduced diameter, and a portion of increased diameter.

A6. The intravascular blood pump of A5, wherein the portion of reduced diameter extends from a point at or substantially near where the catheter is attached to the housing to a point within the restriction element.

A7. The intravascular blood pump of A5, wherein the portion of reduced diameter extends from a point within the restriction element to a point within the proximal bearing.

A8. The intravascular blood pump of A6, wherein the portion of increased diameter extends from a point within the restriction element to a point within the distal bearing.

A9. The intravascular blood pump of A8, wherein the inner layer of wound or braided wires is omitted between a point within the restriction element and a point within the distal bearing.

A10. The intravascular blood pump of A7, wherein the portion of increased diameter extends from a point within the proximal bearing to a point within the distal bearing.

A11. The intravascular blood pump of A10, wherein the inner layer of wound or braided wires is omitted between a point within the proximal bearing and a point within the distal bearing.

A12. The intravascular blood pump of any of A1-A11, wherein the reinforcement element comprises Nitinol or Ultra-Stiff Nitinol.

A13. The intravascular blood pump of any of A1-A12, wherein the housing comprises a cage surrounding the rotor, the cage having a plurality of struts.

A14. The intravascular blood pump of A13, wherein, at a first point proximal of the rotor, each strut of the plurality of struts has a circumferential width and a radial thickness, the circumferential width being between 1.2 and 1.8 times the radial thickness.

A15. The intravascular blood pump of A13, wherein, at a first point proximal of the rotor, each strut of the plurality of struts has a circumferential width and a radial thickness, the circumferential width being between 1.2 and 1.3 times the radial thickness.

A16. The intravascular blood pump of A13, wherein, at a first point proximal of the rotor, each strut of the plurality of struts has a circumferential width and a radial thickness, the circumferential width being about 1.26 times the radial thickness.

A17. The intravascular blood pump of A14, wherein, at a second point distal of the rotor, each strut of the plurality of struts has a circumferential width and a radial thickness, the circumferential width being between 1.2 and 1.8 times the radial thickness.

A18. The intravascular blood pump of A15, wherein, at a second point distal of the rotor, each strut of the plurality of struts has a circumferential width and a radial thickness, the circumferential width being between 1.2 and 1.3 times the radial thickness.

A19. The intravascular blood pump of A16, wherein, at a second point distal of the rotor, each strut of the plurality of struts has a circumferential width and a radial thickness, the circumferential width being about 1.26 times the radial thickness.

A20. The intravascular blood pump of A17, wherein, at a third point proximal of the rotor and distal of the first point, each strut of the plurality of struts has a circumferential width and a radial thickness, the circumferential width being between 1.0 and 1.6 times the radial thickness.

A21. The intravascular blood pump of A18, wherein, at a third point proximal of the rotor and distal of the first point, each strut of the plurality of struts has a circumferential width and a radial thickness, the circumferential width being between 1.0 and 1.15 times the radial thickness.

A22. The intravascular blood pump of A19, wherein, at a third point proximal of the rotor and distal of the first point, each strut of the plurality of struts has a circumferential width and a radial thickness, the circumferential width being about 1.26 times the radial thickness.

A23. The intravascular blood pump of A19, wherein, at a third point proximal of the rotor and distal of the first point, each strut of the plurality of struts has a circumferential width and a radial thickness, the circumferential width being about 1.09 times the radial thickness.

A24. The intravascular blood pump of A20, wherein, at a fourth point distal of the rotor and proximal of the second point, each strut of the plurality of struts has a circumferential width and a radial thickness, the circumferential width being between 1.0 and 1.6 times the radial thickness.

A25. The intravascular blood pump of A21, wherein, at a fourth point distal of the rotor and proximal of the second point, each strut of the plurality of struts has a circumferential width and a radial thickness, the circumferential width being between 1.0 and 1.15 times the radial thickness.

A26. The intravascular blood pump of A22, wherein, at a fourth point distal of the rotor and proximal of the second point, each strut of the plurality of struts has a circumferential width and a radial thickness, the circumferential width being about 1.26 times the radial thickness.

A27. The intravascular blood pump of A23, wherein, at a fourth point distal of the rotor and proximal of the second point, each strut of the plurality of struts has a circumferential width and a radial thickness, the circumferential width being about 1.09 times the radial thickness.

A28. The intravascular blood pump of any of A1-A28, wherein the housing comprises Nitinol or Ultra-Stiff Nitinol.

A29. The intravascular blood pump of A5, wherein the portion of increased diameter is configured to fit within the outer layer of the wound or braided wires in a portion of the drive shaft in which the inner layer of wound or braided wires has been omitted.

A30. The intravascular blood pump of any of A1-A29, further comprising an atraumatic tip at a distal end of the blood pump.

A31. The intravascular blood pump of A30, wherein the predefined bend region of the catheter is configured to make contact with an endothelium of an aorta when the blood pump is inserted into a patient's heart, thereby supporting the pumping device and aligning the atraumatic tip with an aortic valve of the patient's heart and to thereby position the pumping device in a ventricle of the patient's heart.

A32. The intravascular blood pump of A31, wherein the atraumatic tip is between 110 to 140 degrees out of plane with respect to a plane in which the bent sleeve, when bent, lies flat, wherein the atraumatic tip is further optionally 120 to 130 degrees out of plane with respect to a plane in which the bent sleeve, when bent, lies flat, and wherein the atraumatic tip is further optionally 130 degrees out of plane with respect to a plane in which the bent sleeve, when bent, lies flat.

A33. The intravascular blood pump of any of A1-A29, wherein the plurality of openings are formed in radially matched pairs which define an arc or semicircle of about 180 degrees about a circumference of the sleeve.

A34. The intravascular blood pump of A33, wherein each of the openings extends about one-half way around the circumference of the sleeve and each opening having a connector at an opening terminus.

A35. The intravascular blood pump of A34, wherein the radially matched pairs of openings share a common axis and are laterally offset from one another in an alternating fashion.

A36. The intravascular blood pump of any of A1-A29, wherein the plurality of annular rings are spaced apart by a uniform distance when the sleeve is in a straight configuration.

A37. The intravascular blood pump of any of A1-A29, wherein a length of the sleeve corresponds to a length of the predefined bend region on the catheter.

A38. The intravascular blood pump of any of A1-A29, further comprising a strain relief section at a distal and/or proximal end of the sleeve.

A39. The intravascular blood pump of A38, wherein the strain relief section includes a stiffness that is different from a rest of the sleeve.

A40. The intravascular blood pump of A39, wherein the strain relief section includes one or more struts.

A41. The intravascular blood pump of A40, where the one or more struts include one or more spiral struts.

A42. The intravascular blood pump of A39, wherein a shape of a pattern can be formed via a wind-up of a flat pattern.

Category B:

B1. An intravascular blood pump, comprising:
a catheter;
a housing in which a rotor is housed, the housing being attached to a distal end of the catheter; and
a drive shaft extending through the catheter and connected to the rotor, the drive shaft comprising an outer layer of wound or braided wires, an inner layer of wound or braided wires, and a reinforcement element arranged within at least the outer layer of wound or braided wires,
wherein the drive shaft is rotatably supported in a proximal bearing located proximal of the rotor and a distal bearing located distal of the rotor, and
wherein the reinforcement element extends from at least a point within the proximal bearing to a point within the distal bearing
wherein the catheter comprises a sleeve configured to control a position of the pumping device in a patient's heart, the sleeve comprising:
a plurality of annular rings;
at least two connectors, the at least two connectors disposed between each annular ring for connecting each of the plurality of annular rings, the at least two connectors being offset from adjacent connectors; and
a plurality of openings formed between each ring,
wherein the sleeve is configured to be monolithically integrated with or placed over the predefined bend region of the catheter and thereby provide a predefined resilient bend in the catheter at the predefined bend region.

B2. The intravascular blood pump of B1, wherein the reinforcement element extends from a point proximal to the proximal bearing to a point within the distal bearing.

B3. The intravascular blood pump of B1 or B2, wherein the proximal bearing comprises a bearing sleeve attached to the drive shaft and an outer bearing ring attached to the housing, the bearing sleeve being configured to rotate within the outer bearing ring.

B4. The intravascular blood pump of B3, further comprising a restriction element attached to the housing and located proximal of the proximal bearing and configured to prevent the bearing sleeve from becoming dislodged from the outer bearing ring.

B5. The intravascular blood pump of any of B1 to B4, wherein the reinforcement element comprises a stepped proximal end with a portion of reduced diameter, and a portion of increased diameter.

B6. The intravascular blood pump of B5, wherein the portion of reduced diameter extends from a point substantially near where the catheter is attached to the housing to a point within the restriction element.

B7. The intravascular blood pump of B5 or B6, wherein the portion of reduced diameter extends from a point within the restriction element to a point within the proximal bearing.

B8. The intravascular blood pump of any of B5 to B7, wherein the portion of increased diameter extends from a point within the restriction element to a point within the distal bearing.

B9. The intravascular blood pump of any of B1 to B8, wherein the inner layer of wound or braided wires is omitted between a point within the restriction element and a point within the distal bearing.

B10. The intravascular blood pump of any of B1 to B9, wherein the portion of increased diameter extends from a point within the proximal bearing to a point within the distal bearing.

B11. The intravascular blood pump of any one of B1 to B10, wherein the portion of increased diameter is configured to fit within the outer layer of the drive shaft in a portion of the drive shaft in which the inner layer has been omitted.

B12. The intravascular blood pump of any one of B1 to B11, wherein the inner layer of wound or braided wires is omitted between a point within the proximal bearing and a point within the distal bearing.

B13. The intravascular blood pump of any of B1 to B12, wherein the reinforcement element comprises Nitinol or Ultra-Stiff Nitinol.

B14. The intravascular blood pump of any of B1 to B13, wherein the housing comprises a cage surrounding the rotor, the cage having a plurality of struts.

B15. The intravascular blood pump of B14, wherein, at a first point proximal of the rotor, each strut of the plurality of struts has a circumferential width and a radial thickness, the circumferential width being about 1.26 times the radial thickness.

B16. The intravascular blood pump of B14 or B15, wherein, at a second point distal of the rotor, each strut of the plurality of struts has a circumferential width and a radial thickness, the circumferential width being about 1.26 times the radial thickness.

B17. The intravascular blood pump of any of B14 to B16, wherein, at a third point proximal of the rotor and distal of the first point, each strut of the plurality of struts has a circumferential width and a radial thickness, the circumferential width being about 1.26 times the radial thickness.

B18. The intravascular blood pump of any of B14 to B17, wherein, at a fourth point distal of the rotor and proximal of the second point, each strut of the plurality of struts has a circumferential width and a radial thickness, the circumferential width being about 1.26 times the radial thickness.

B19. The intravascular blood pump of any of B14 to B18, wherein, at a third point proximal of the rotor and distal of the first point, each strut of the plurality of struts has a circumferential width and a radial thickness, the circumferential width being about 1.09 times the radial thickness.

B20. The intravascular blood pump of any of B14 to B19, wherein, at a fourth point distal of the rotor and proximal of the second point, each strut of the plurality of struts has a circumferential width and a radial thickness, the circumferential width being about 1.09 times the radial thickness.

B21. The intravascular blood pump of any of B1 to B20, wherein at least one of the rotor and the housing comprises Nitinol or Ultra-Stiff Nitinol.

B22. The intravascular blood pump of any of B1 to B21, wherein the intravascular blood pump comprises a pump section, wherein the pump section comprises the rotor.

B23. The intravascular blood pump of B22, wherein the rotor is configured to cause blood to flow from a blood flow inlet at a distal end of the pump section to a blood flow outlet located proximally of the blood flow inlet.

B24. The intravascular blood pump of B22 or B23, wherein the pump section comprises the housing.

B25. The intravascular blood pump of any of B1 to B24, wherein at least one of the rotor and the housing are compressible, such that the intravascular blood pump may be inserted through a patient's vascular system into the patient's heart while at least one of the rotor and the housing are in their compressed state, and such that the rotor and housing may be expanded once the pump section is positioned at its target location.

B26. The intravascular blood pump of any of B1 to B25, wherein the reinforcement element is a solid rod or wire.

B27. The intravascular blood pump of any of B1 to B26, wherein the reinforcement element is arranged coaxially within the drive shaft.

B28. The intravascular blood pump of any of B1 to B27, wherein the drive shaft and/or the reinforcement element is hollow along some or all of its length.

B29. The intravascular blood pump of any of B1 to B28, wherein the distal bearing includes an outer sleeve which houses a spiral bearing.

B30. The intravascular blood pump of B29, wherein the spiral bearing is configured to surround the drive shaft.

B31. The intravascular blood pump of any of B1 to B28, further comprising an atraumatic tip at a distal end of the blood pump.

B32. The intravascular blood pump of B31, wherein the predefined bend region of the catheter is configured to make contact with an endothelium of an aorta when the blood pump is inserted into a patient's heart, thereby supporting the pumping device and aligning the atraumatic tip with an aortic valve of the patient's heart and to thereby position the pumping device in a ventricle of the patient's heart.

B33. The intravascular blood pump of B32, wherein the predefined bend region of the catheter is adapted to make contact with an endothelium of an aorta when the blood pump is inserted into a patient's heart, thereby supporting the pumping device and aligning the atraumatic tip with an aortic valve of the patient's heart and to thereby position the pumping device in a ventricle of the patient's heart.

B34. The intravascular blood pump of B33, wherein the atraumatic tip is between 110 to 140 degrees out of plane with respect to a plane in which the bent sleeve, when bent, lies flat, wherein the atraumatic tip is further optionally 120 to 130 degrees out of plane with respect to a plane in which the bent sleeve, when bent, lies flat, and wherein the atraumatic tip is further optionally 130 degrees out of plane with respect to a plane in which the bent sleeve, when bent, lies flat.

B35. The intravascular blood pump of any of B1 to B28, wherein the plurality of openings are formed in radially matched pairs which define an arc or semicircle of about 180 degrees about a circumference of the sleeve.

B36. The intravascular blood pump of B35, wherein each of the openings extends about one-half way around the circumference of the sleeve and each opening having a connector at an opening terminus.

B37. The intravascular blood pump of B36, wherein the radially matched pairs of openings share a common axis and are laterally offset from one another in an alternating fashion.

B38. The intravascular blood pump of any of B1 to B28, wherein the plurality of annular rings are spaced apart by a uniform distance when the sleeve is in a straight configuration.

B39. The intravascular blood pump of any of B1 to B28, wherein a length of the sleeve corresponds to a length of the predefined bend region on the catheter.

Category C:

C1. An intravascular blood pump, comprising:
a catheter;
a housing in which a rotor is housed, the housing being attached to a distal end of the catheter; and
a drive shaft extending through the catheter and connected to the rotor, at least a portion of the drive shaft being flexible, the drive shaft comprising an outer layer of wound or braided wires, an inner layer of wound or braided wires, and a reinforcement element arranged within at least the outer layer of wound or braided wires,
wherein the drive shaft is rotatably supported in a proximal bearing located proximal of the rotor and a distal bearing located distal of the rotor, wherein the reinforcement element extends from at least a point within the proximal bearing to a point within the distal bearing wherein a catheter having a distal end and a predefined bend region positioned proximal to the distal end;
wherein the catheter comprises a sleeve comprising:
a plurality of annular rings;
at least two connectors disposed between each of the plurality of annular rings for connecting each of the plurality of annular rings, the at least two connectors being offset from at least one adjacent connector; and
a plurality of openings formed between each annular ring and arranged in an alternating repeating fashion,
wherein the sleeve is configured to be monolithically integrated with or placed over a predefined bend region of a catheter and thereby provide a predefined resilient bend in the catheter.
   C2. The intravascular blood pump of C1, further comprising a strain relief region at a proximal and/or distal end of the sleeve.

Category D:

D1. An intravascular blood pump, comprising:
a catheter;
a housing in which a rotor is housed, the housing being attached to a distal end of the catheter; and
a drive shaft extending through the catheter and connected to the rotor, the drive shaft comprising an outer layer of wound or braided wires, an inner layer of wound or braided wires, and a reinforcement element arranged within at least the outer layer of wound or braided wires,
wherein the drive shaft is rotatably supported in a proximal bearing located proximal of the rotor and a distal bearing located distal of the rotor, and
wherein the reinforcement element extends from at least a point within the proximal bearing to a point within the distal bearing;
the catheter comprising a sleeve comprising:
a plurality of annular rings;
at least two connectors disposed between each of the plurality of annular rings for connecting each of the plurality of annular rings, the at least two connectors being offset from at least one adjacent connector; and
a plurality of openings formed between each annular ring and arranged in an alternating repeating fashion,
wherein the sleeve is configured to be monolithically integrated with or placed over a predefined bend region of a catheter and thereby provide a predefined resilient bend in the catheter.

Category E:

E1. An intravascular blood pump, comprising:
a catheter;
a housing in which a rotor is housed, the housing being attached to a distal end of the catheter; and
a drive shaft extending through the catheter and connected to the rotor, at least a portion of the drive shaft being flexible, the drive shaft comprising an outer layer of wound or braided wires, an inner layer of wound or braided wires, and a reinforcement element arranged within at least the outer layer of wound or braided wires,
wherein the drive shaft is rotatably supported in a proximal bearing located proximal of the rotor and a distal bearing located distal of the rotor, and
wherein the reinforcement element extends from at least a point within the proximal bearing to a point within the distal bearing.
   E2. The intravascular blood pump of E1, wherein the reinforcement element extends from a point proximal to the proximal bearing to a point within the distal bearing.
   E3. The intravascular blood pump of any of E1-E2, wherein the proximal bearing comprises a bearing sleeve attached to the drive shaft and an outer bearing ring attached to the housing, the bearing sleeve being configured to rotate within the outer bearing ring.
   E4. The intravascular blood pump of E3, further comprising a restriction element attached to the housing and located proximal of the proximal bearing and configured to prevent the bearing sleeve from becoming dislodged from the outer bearing ring.
   E5. The intravascular blood pump of any of E1-E4, wherein the reinforcement element comprises a stepped proximal end with a portion of reduced diameter, and a portion of increased diameter.
   E6. The intravascular blood pump of E5, wherein the portion of reduced diameter extends from a point at or substantially near where the catheter is attached to the housing to a point within the restriction element.
   E7. The intravascular blood pump of E5, wherein the portion of reduced diameter extends from a point within the restriction element to a point within the proximal bearing.
   E8. The intravascular blood pump of E6, wherein the portion of increased diameter extends from a point within the restriction element to a point within the distal bearing.
   E9. The intravascular blood pump of E5, wherein the inner layer of wound or braided wires is omitted between a point within the restriction element and a point within the distal bearing.
   E10. The intravascular blood pump of E7, wherein the portion of increased diameter extends from a point within the proximal bearing to a point within the distal bearing.
   E11. The intravascular blood pump of E10, wherein the inner layer of wound or braided wires is omitted between a point within the proximal bearing and a point within the distal bearing.
   E12. The intravascular blood pump of any of E1-E11, wherein the reinforcement element comprises Nitinol or Ultra-Stiff Nitinol.
   E13. The intravascular blood pump of any of E1-E12, wherein the housing comprises a cage surrounding the rotor, the cage having a plurality of struts.
   E14. The intravascular blood pump of E13, wherein, at a first point proximal of the rotor, each strut of the plurality of struts has a circumferential width and a radial thickness, the circumferential width being between 1.2 and 1.8 times the radial thickness.
   E15. The intravascular blood pump of E13, wherein, at a first point proximal of the rotor, each strut of the plurality of struts has a circumferential width and a radial thickness, the circumferential width being between 1.2 and 1.3 times the radial thickness.
   E16. The intravascular blood pump of E13, wherein, at a first point proximal of the rotor, each strut of the plurality of struts has a circumferential width and a radial thickness, the circumferential width being about 1.26 times the radial thickness.
   E17. The intravascular blood pump of E14 wherein, at a second point distal of the rotor, each strut of the plurality of struts has a circumferential width and a radial thickness, the circumferential width being between 1.2 and 1.8 times the radial thickness.
   E18. The intravascular blood pump of E15, wherein, at a second point distal of the rotor, each strut of the plurality of struts has a circumferential width and a radial thickness, the circumferential width being between 1.2 and 1.3 times the radial thickness.
   E19. The intravascular blood pump of E16, wherein, at a second point distal of the rotor, each strut of the plurality of struts has a circumferential width and a radial thickness, the circumferential width being about 1.26 times the radial thickness.
   E20. The intravascular blood pump of E17, wherein, at a third point proximal of the rotor and distal of the first point, each strut of the plurality of struts has a circumferential width and a radial thickness, the circumferential width being between 1.0 and 1.6 times the radial thickness.
   E21. The intravascular blood pump of E18, wherein, at a third point proximal of the rotor and distal of the first point, each strut of the plurality of struts has a circumferential width and a radial thickness, the circumferential width being between 1.0 and 1.15 times the radial thickness.
   E22. The intravascular blood pump of E19, wherein, at a third point proximal of the rotor and distal of the first point, each strut of the plurality of struts has a circumferential width and a radial thickness, the circumferential width being about 1.26 times the radial thickness.
   E23. The intravascular blood pump of E19, wherein, at a third point proximal of the rotor and distal of the first point, each strut of the plurality of struts has a circumferential width and a radial thickness, the circumferential width being about 1.09 times the radial thickness.
   E24. The intravascular blood pump of E20, wherein, at a fourth point distal of the rotor and proximal of the second point, each strut of the plurality of struts has a circumferential width and a radial thickness, the circumferential width being between 1.0 and 1.6 times the radial thickness.
   E25. The intravascular blood pump of E21, wherein, at a fourth point distal of the rotor and proximal of the second point, each strut of the plurality of struts has a circumferential width and a radial thickness, the circumferential width being between 1.0 and 1.15 times the radial thickness.
   E26. The intravascular blood pump of E22, wherein, at a fourth point distal of the rotor and proximal of the second point, each strut of the plurality of struts has a circumferential width and a radial thickness, the circumferential width being about 1.26 times the radial thickness.
   E27. The intravascular blood pump of E23, wherein, at a fourth point distal of the rotor and proximal of the second point, each strut of the plurality of struts has a circumferential width and a radial thickness, the circumferential width being about 1.09 times the radial thickness.
   E28. The intravascular blood pump of any of E1-E27, wherein the housing comprises Nitinol or Ultra-Stiff Nitinol.
   E29. The intravascular blood pump of E5, wherein the portion of increased diameter is configured to fit within the outer layer of the wound or braided wires in a portion of the drive shaft in which the inner layer of wound or braided wires has been omitted.
   E30. The intravascular blood pump of E1, further comprising a downstream tubing attached to the housing and through which the catheter is disposed, wherein the downstream tubing is bent.
   E31. The intravascular blood pump of E30, wherein the downstream tubing in made of a flexible material such that it may be compressed or expanded.
   E32. The intravascular blood pump of E31, wherein a bend angle of the downstream tubing is different than a bend angle of the catheter.
   E33. The intravascular blood pump of E32, wherein the bend angle of the downstream tubing is 30°±10° and the bend angle of the catheter is 45°±10°.
   E34. The intravascular blood pump of E30, wherein a bend angle of the downstream tubing and a bend angle of the catheter is the same.

Category F:

F1. An intravascular blood pump, comprising:
a catheter;
a housing in which a rotor is housed, the housing being attached to a distal end of the catheter; and
a drive shaft extending through the catheter and connected to the rotor, the drive shaft comprising an outer layer of wound or braided wires, an inner layer of wound or braided wires, and a reinforcement element arranged within at least the outer layer of wound or braided wires, wherein the drive shaft is rotatably supported in a proximal bearing located proximal of the rotor and a distal bearing located distal of the rotor, and
wherein the reinforcement element extends from at least a point within the proximal bearing to a point within the distal bearing.
   F2. The intravascular blood pump of F1, wherein the reinforcement element extends from a point proximal to the proximal bearing to a point within the distal bearing.
   F3. The intravascular blood pump of F1 or F2, wherein the proximal bearing comprises a bearing sleeve attached to the drive shaft and an outer bearing ring attached to the housing, the bearing sleeve being configured to rotate within the outer bearing ring.
   F4. The intravascular blood pump of F3, further comprising a restriction element attached to the housing and located proximal of the proximal bearing and configured to prevent the bearing sleeve from becoming dislodged from the outer bearing ring.
   F5. The intravascular blood pump of any of F1 to F4, wherein the reinforcement element comprises a stepped proximal end with a portion of reduced diameter, and a portion of increased diameter.
   F6. The intravascular blood pump of F5, wherein the portion of reduced diameter extends from a point substantially near where the catheter is attached to the housing to a point within the restriction element.
   F7. The intravascular blood pump of F5 or F6, wherein the portion of reduced diameter extends from a point within the restriction element to a point within the proximal bearing.
   F8. The intravascular blood pump of any of F5 to F7, wherein the portion of increased diameter extends from a point within the restriction element to a point within the distal bearing.
   F9. The intravascular blood pump of any of F1 to F8, wherein the inner layer of wound or braided wires is omitted between a point within the restriction element and a point within the distal bearing.
   F10. The intravascular blood pump of any of F1 to F9, wherein the portion of increased diameter extends from a point within the proximal bearing to a point within the distal bearing.
   F11. The intravascular blood pump of any one of F1 to F10, wherein the portion of increased diameter is configured to fit within the outer layer of the drive shaft in a portion of the drive shaft in which the inner layer has been omitted.
   F12. The intravascular blood pump of any one of F1 to F11, wherein the inner layer of wound or braided wires is omitted between a point within the proximal bearing and a point within the distal bearing.
   F13. The intravascular blood pump of any of F1 to F12, wherein the reinforcement element comprises Nitinol or Ultra-Stiff Nitinol.
   F14. The intravascular blood pump of any of F1 to F13, wherein the housing comprises a cage surrounding the rotor, the cage having a plurality of struts.
   F15. The intravascular blood pump of F14, wherein, at a first point proximal of the rotor, each strut of the plurality of struts has a circumferential width and a radial thickness, the circumferential width being about 1.26 times the radial thickness.
   F16. The intravascular blood pump of F14 or F15, wherein, at a second point distal of the rotor, each strut of the plurality of struts has a circumferential width and a radial thickness, the circumferential width being about 1.26 times the radial thickness.
   F17. The intravascular blood pump of any of F14 to F16, wherein, at a third point proximal of the rotor and distal of the first point, each strut of the plurality of struts has a circumferential width and a radial thickness, the circumferential width being about 1.26 times the radial thickness.
   F18. The intravascular blood pump of any of F14 to F17, wherein, at a fourth point distal of the rotor and proximal of the second point, each strut of the plurality of struts has a circumferential width and a radial thickness, the circumferential width being about 1.26 times the radial thickness.
   F19. The intravascular blood pump of any of F14 to F18, wherein, at a third point proximal of the rotor and distal of the first point, each strut of the plurality of struts has a circumferential width and a radial thickness, the circumferential width being about 1.09 times the radial thickness.
   F20. The intravascular blood pump of any of F14 to F19, wherein, at a fourth point distal of the rotor and proximal of the second point, each strut of the plurality of struts has a circumferential width and a radial thickness, the circumferential width being about 1.09 times the radial thickness.
   F21. The intravascular blood pump of any of F1 to F20, wherein at least one of the rotor and the housing comprises Nitinol or Ultra-Stiff Nitinol.
   F22. The intravascular blood pump of any of F1 to F21, wherein the intravascular blood pump comprises a pump section, wherein the pump section comprises the rotor.
   F23. The intravascular blood pump of F22, wherein the rotor is configured to cause blood to flow from a blood flow inlet at a distal end of the pump section to a blood flow outlet located proximally of the blood flow inlet.
   F24. The intravascular blood pump of F22 or F23, wherein the pump section comprises the housing.
   F25. The intravascular blood pump of any of B1 to B24, wherein at least one of the rotor and the housing are compressible, such that the intravascular blood pump can be inserted through a patient's vascular system into the patient's heart while at least one of the rotor and the housing are in their compressed state, and such that the rotor and housing may be expanded once the pump section is positioned at its target location.
   F26. The intravascular blood pump of any of F1 to F25, wherein the reinforcement element is a solid rod or wire.
   F27. The intravascular blood pump of any of F1 to F26, wherein the reinforcement element is arranged coaxially within the drive shaft.
   F28. The intravascular blood pump of any of F1 to F27, wherein the drive shaft and/or the reinforcement element is hollow along some or all of its length.
   F29. The intravascular blood pump of any of F1 to F28, wherein the distal bearing includes an outer sleeve which houses a spiral bearing.
   F30. The intravascular blood pump of F29, wherein the spiral bearing is configured to surround the drive shaft.
   F31. The intravascular blood pump of F1, wherein the catheter includes a bent catheter.
   F31. The intravascular blood pump of F31, further comprising a downstream tubing attached to the housing and through which the catheter is disposed, wherein the downstream tubing is bent.
   F32. The intravascular blood pump of F31, wherein the downstream tubing in made of a flexible material such that it may be compressed or expanded.
   F33. The intravascular blood pump of F31, wherein a bend angle of the downstream tubing is different than a bend angle of the catheter.
   F34. The intravascular blood pump of F33, wherein the bend angle of the downstream tubing is 30°±10° and the bend angle of the catheter is 45°±10°.
   F35. The intravascular blood pump of F31, wherein a bend angle of the downstream tubing and a bend angle of the catheter is the same.

1. An intravascular blood pump, comprising:
a catheter;
a housing in which a rotor is housed, the housing being attached to a distal end of the catheter; and
a drive shaft extending through the catheter and connected to the rotor, at least a portion of the drive shaft being flexible, the drive shaft comprising an outer layer of wound or braided wires, an inner layer of wound or braided wires, and a reinforcement element arranged within at least the outer layer of wound or braided wires,
wherein the drive shaft is rotatably supported in a proximal bearing located proximal of the rotor and a distal bearing located distal of the rotor, and
wherein the reinforcement element extends from at least a point within the proximal bearing to a point within the distal bearing.

2. The intravascular blood pump of claim 1, wherein the reinforcement element extends from a point proximal to the proximal bearing to a point within the distal bearing.

3. The intravascular blood pump of claim 1, wherein the proximal bearing comprises a bearing sleeve attached to the drive shaft and an outer bearing ring attached to the housing, the bearing sleeve being configured to rotate within the outer bearing ring.

4. The intravascular blood pump of claim 3, further comprising a restriction element attached to the housing and located proximal of the proximal bearing and configured to prevent the bearing sleeve from becoming dislodged from the outer bearing ring.

5. The intravascular blood pump of claim 1, wherein the reinforcement element comprises a stepped proximal end with a portion of reduced diameter, and a portion of increased diameter.

6. The intravascular blood pump of claim 5, wherein the portion of reduced diameter extends from a point at or substantially near where the catheter is attached to the housing to a point within the restriction element.

7. The intravascular blood pump of claim 5, wherein the portion of reduced diameter extends from a point within the restriction element to a point within the proximal bearing.

8. The intravascular blood pump of claim 6, wherein the portion of increased diameter extends from a point within the restriction element to a point within the distal bearing.

9. The intravascular blood pump of claim 5, wherein the inner layer of wound or braided wires is omitted between a point within the restriction element and a point within the distal bearing.

10. The intravascular blood pump of claim 7, wherein the portion of increased diameter extends from a point within the proximal bearing to a point within the distal bearing.

11. The intravascular blood pump of claim 10, wherein the inner layer of wound or braided wires is omitted between a point within the proximal bearing and a point within the distal bearing.

12. The intravascular blood pump of claim 1, wherein the reinforcement element comprises Nitinol or Ultra-Stiff Nitinol.

13. The intravascular blood pump of claim 1, wherein the housing comprises a cage surrounding the rotor, the cage having a plurality of struts.

14-27. (canceled)

28. The intravascular blood pump of claim 1, wherein the housing comprises Nitinol or Ultra-Stiff Nitinol.

29. The intravascular blood pump of claim 5, wherein the portion of increased diameter is configured to fit within the outer layer of the wound or braided wires in a portion of the drive shaft in which the inner layer of wound or braided wires has been omitted.

30. The intravascular blood pump of claim 1, further comprising a downstream tubing attached to the housing and through which the catheter is disposed, wherein the downstream tubing is bent.

31. The intravascular blood pump of claim 30, wherein the downstream tubing is made of a flexible material such that it may be compressed or expanded.

32. The intravascular blood pump of claim 31, wherein a bend angle of the downstream tubing is different than a bend angle of the catheter.

33. The intravascular blood pump of claim 32, wherein the bend angle of the downstream tubing is 30°±10° and the bend angle of the catheter is 45°±10°.

34. The intravascular blood pump of claim 30, wherein a bend angle of the downstream tubing and a bend angle of the catheter is the same.

35. An intravascular blood pump, comprising:
a catheter;
a housing in which a rotor is housed, the housing being attached to a distal end of the catheter; and
a drive shaft extending through the catheter and connected to the rotor, the drive shaft comprising an outer layer of wound or braided wires, an inner layer of wound or braided wires, and a reinforcement element arranged within at least the outer layer of wound or braided wires, wherein the drive shaft is rotatably supported in a proximal bearing located proximal of the rotor and a distal bearing located distal of the rotor, and
wherein the reinforcement element extends from at least a point within the proximal bearing to a point within the distal bearing.

36-91. (canceled)
